(19) United States
(12) Patent Application Publication
(10) Pub. No.: US 2023/0212500 A1
(43) Pub. Date: Jul. 6, 2023

(54) GAS AND LIQUID FLOW REGULATION SYSTEM FOR CELL CULTURE

(71) Applicant: Xcell Biosciences, Inc., San Francisco, CA (US)

(72) Inventors: Nicholas George ERNDT, Los Altos Hills, CA (US); Genevieve PEDROTTI, Soquel, CA (US); James LIM, San Francisco, CA (US); Brian FETH, San Francisco, CA (US); Quynh Anh Thuc LU, Mountain View, CA (US)

(21) Appl. No.: 17/976,331

(22) Filed: Oct. 28, 2022

Related U.S. Application Data
(63) Continuation of application No. PCT/US2021/017877, filed on Feb. 12, 2021.
(60) Provisional application No. 62/976,690, filed on Feb. 14, 2020.

(51) Int. Cl. C12M 1/34; C12M 1/107

(52) U.S. Cl. CPC C12M 41/40; C12M 41/34; C12M 41/44; C12M 23/36

(57) ABSTRACT

Embodiments of the provided technology relate to a gas flow and liquid flow regulation system within a cell culture instrument. Embodiments of the gas and liquid flow regulation system include a pressurizable gas-mixing chamber, a cell culture compartment that includes a cell culture vessel having and a gas space, and a gas flow system. The gas flow system is driven by gas pressure, and is adaptable to provide an atmospheric condition of hypoxia and hyperbaric pressure within the cell culture compartment. The liquid flow regulation system is driven by hydraulic pressure.

### CROSS REFERENCE

This application is a continuation of PCT/US21/17877 filed Feb. 12, 2021, which claims the benefit of U.S. Provisional Application No. 62/976,690, filed Feb. 14, 2020, which is incorporated herein by reference in its entirety.

### TECHNICAL FIELD

This application is generally directed to systems for cell culture. More particularly, this application is directed to systems and methods of cell culture that control oxygen level and atmospheric pressure within a cell culture compartment.

### BACKGROUND

The use of human cells, particularly immune cells, as therapeutic agents is a rapidly expanding area of modern medicine. One example is for the manufacture of chimeric antigen receptor expressing T cells (CAR-T) for use in anti-cancer immunotherapy regimens. In accordance with these developments, there is a demand for new technologies that are rigorous, controllable, consistent, and provide high yield in a clinical manufacturing workflow.

### SUMMARY OF THE TECHNOLOGY

The application provides various embodiments of a cell culture technology, including (1) a gas and liquid flow regulation system, (2) a method of regulating gas flow, (3) a method of regulating gas and liquid flow, (4) a method of expanding a cell population, and (5) a cell culture instrument.

In a first embodiment of the technology, a gas flow and liquid flow regulation system for a cell culture instrument is provided. The cell culture instrument includes a pressurizable gas-mixing chamber a pressurizable gas-mixing chamber having operable connections to multiple gas sources; a cell culture compartment including (a) a cell culture vessel that can hold a liquid medium for cell culture and (b) a gas space, wherein the liquid medium and the gas space meet at an interface; and a gas flow system that includes a circular flow path portion, the circular portion including (a) a first gas flow path segment from the pressurizable gas-mixing chamber to the cell culture compartment; (b) a second gas flow path segment from cell culture compartment back to the pressurizable gas-mixing chamber, wherein the gas flow system is adaptable to provide a hyperbaric and hypoxic atmospheric condition within the cell culture compartment.

In a second embodiment of the technology, a method of regulating gas flow and liquid flow within a gas and liquid flow regulation system of a cell culture compartment of a cell culture instrument is provided. This method includes forming a gas-phase gas composition, as desired, in a pressurizable gas-mixing chamber, wherein the gas-phase gas composition has a hyperbaric atmospheric pressure and a hypoxic oxygen partial pressure; conveying the gas-phase gas composition into the cell culture compartment, wherein the cell culture compartment contains a liquid culture medium; within the cell culture compartment, interfacing the liquid culture medium with the gas-phase composition in the cell culture compartment to allow a dissolved gas composition in the liquid medium to approach equilibrium with the gas-phase gas composition; and circulating the hyperbaric and hypoxic gas composition through a circular portion of a gas flow path that includes the cell culture compartment and the pressurizable gas mixing chamber.

In a third embodiment of the technology, a method of expanding a cell population in a cell culture compartment of a cell culture instrument is provided. This method includes forming a hyperbaric and hypoxic gas-phase gas composition in a pressurizable gas-mixing chamber of the instrument, wherein the instrument further includes a cell culture compartment having a cell culture vessel disposed within a gas space, and wherein the vessel is holding a volume of liquid cell culture medium; flowing the gas-phase gas composition from the pressurizable gas-mixing chamber into the gas space of the cell culture compartment, thereby interfacing the liquid culture medium in the cell culture bag with the gas-phase composition in the gas space so as to allow a dissolved gas composition in the liquid medium to approach equilibrium with the gas-phase gas composition; effluxing the hyperbaric and hypoxic gas-phase gas composition from the gas space and conveying the gas-phase gas composition back into the pressurizable gas-mixing chamber, thereby establishing a circular gas flow loop; inoculating an initial cell population into a gas permeable cell culture bag containing liquid cell culture medium, wherein the cell culture bag is disposed within a cell culture cartridge; flowing an amount of fresh cell culture medium into the cell culture bag and flowing a substantially equivalent amount of cell-conditioned cell culture medium out of the cell culture bag; circulating the hyperbaric and hypoxic gas composition through a circular portion of a gas flow path that comprises the cell culture compartment and the pressurizable gas mixing chamber; and culturing the initial cell population over a cell culture duration to provide an expanded cell population.

In a fourth embodiment of the technology, a gas flow and liquid flow regulation system for a cell culture instrument is provided. This system includes a pressurizable gas-mixing chamber comprising multiple gas injection ports operably connected to multiple gas sources; a cell culture compartment having (a) a cell culture vessel that can hold a liquid medium for cell culture and (b) a gas space, wherein the liquid medium and the gas space meet at an interface; a gas flow system comprising a circular flow path portion, the circular portion comprising (a) a first gas flow path segment from the pressurizable gas-mixing chamber to the cell culture compartment; (b) a second gas flow path segment from cell culture compartment back to the pressurizable gas-mixing chamber, wherein the gas flow system is adaptable to provide a hyperbaric and hypoxic atmospheric condition within the cell culture compartment, and a liquid flow regulation system comprising a liquid flow path comprising (a) a liquid flow path segment from the liquid cell culture medium source container into the cell culture compartment; (b) a liquid flow path segment from the inoculum source container into the cell culture compartment; and (c) a liquid flow path segment from the cell culture compartment into a downstream cell culture collection container, wherein the flow path comprises a perfusion cell culture process.

In a fifth embodiment of the technology, an instrument for cell culture and a gas and liquid flow regulation system is provided. This instrument includes a housing; a temperature controlled incubator disposed within the housing; a gas flow and liquid flow regulation system for a cell culture instrument disposed within the incubator, a pressurizable gas-mixing chamber disposed within the incubator; a cell culture compartment disposed within the incubator, the compartment comprising (a) a cell culture vessel that can hold a liquid medium for cell culture and (b) a gas space, wherein the liquid medium and the gas space meet at an interface; and a gas flow system disposed within the incubator, the gas flow system comprising a circular flow path portion disposed within the cell culture compartment, the circular portion comprising (a) a first gas flow path segment from the pressurizable gas-mixing chamber to the cell culture compartment; (b) a second gas flow path segment from cell culture compartment back to the pressurizable gas-mixing chamber, wherein the gas flow system is adaptable to provide a hyperbaric and hypoxic atmospheric condition within the cell culture compartment.

The instrument of the fifth embodiment further includes a liquid flow regulation system disposed within the incubator, the liquid flow system comprising a liquid flow path, the flow path including (a) a liquid flow path segment from the liquid cell culture medium source container into the cell culture compartment; (b) a liquid flow path segment from the inoculum source container into the cell culture compartment; and (c) a liquid flow path segment from the cell culture compartment into a downstream cell culture collection container.

### BRIEF DESCRIPTION OF THE FIGURES

FIG. 1A is a block diagram of an embodiment of a gas and liquid flow regulation system for cell culture, with a focus on the gas flow aspect of the system.

FIG. 1B is a block diagram of an embodiment of a gas and liquid flow regulation system for cell culture, with a focus on the liquid flow aspect of the system.

FIG. 1C is a block diagram of an embodiment of a gas and liquid flow regulation system for cell culture, including the combined gas flow and liquid flow aspects of the system.

FIG. 1D is a block diagram of an embodiment of a gas and liquid flow regulation system for cell culture, with a focus on gas-based and liquid-based sensors within the system.

FIG. 1E is a block diagram of an embodiment of a gas and liquid flow regulation system for cell culture, with a focus on gas and liquid pumps disposed within the gas and liquid flow paths.

FIG. 1F is a block diagram of an embodiment of a gas and liquid flow regulation system for cell culture as disposed within cell culture incubator, a temperature controlled portion of the system.

FIG. 1G is a schematic diagram of a cell culture compartment embodiment of a gas and liquid flow regulation system for cell culture disposed within cell culture incubator, a temperature controlled portion of the system.

FIG. 2A is a perspective view of an embodiment of a cell culture instrument that houses a gas and liquid flow regulation system.

FIG. 2B is a perspective view of an embodiment of a cell culture instrument that houses a gas and liquid flow regulation system; the front door is open and the top removed to provide a view into the interior of the instrument.

FIG. 2C is a top view of an embodiment of a cell culture instrument that houses a gas and liquid flow regulation system; the top is removed to provide a view into the interior of the instrument.

FIG. 2D is a top view of an embodiment of a cell culture instrument that houses a gas and liquid flow regulation system; the front door is open and the top removed to provide a view into the interior of the instrument; a cell culture cartridge is shown outside the instrument, aligned with a receptacle within the instrument.

FIG. 2E is a schematic diagram that shows details of a system of liquid flow within a cell culture instrument.

FIGS. 3A-3G are various views of a cell culture compartment; in this embodiment, the cell culture compartment is configured as cylindrical cartridge.

FIG. 3A is an isometric perspective view of a cell culture cartridge for an embodiment of a gas and liquid flow regulation system for cell culture, showing the flow paths of gas and liquid there through.

FIG. 3B is a side view of a cell culture cartridge for an embodiment of a gas and liquid flow regulation system for cell culture.

FIG. 3C is a top view of a cell culture cartridge for an embodiment of a gas and liquid flow regulation system for cell culture.

FIG. 3D is a cross-sectional view of a cell culture cartridge for an embodiment of a gas and liquid flow regulation system for cell culture.

FIG. 3E is a face view of the front lid of cell culture cartridge for an embodiment of a gas and liquid flow regulation system for cell culture.

FIG. 3F is a face view of the back lid of cell culture cartridge for an embodiment of a gas and liquid flow regulation system for cell culture.

FIG. 3G is a length-wise cross-sectional view of the front end of a cell culture cartridge and a connector for gas and fluid flow for an embodiment of a gas and liquid flow regulation system for cell culture.

FIG. 4A is a block diagram of an alternative embodiment of a gas and liquid flow regulation system for cell culture, with a focus on the gas flow aspect of the system.

FIG. 4B is a block diagram of alternative embodiment of a gas and liquid flow regulation system for cell culture, with a focus on the liquid flow aspect of the system.

FIG. 4C is a block diagram of alternative embodiment of a gas and liquid flow regulation system for cell culture, including the combined gas flow and liquid flow aspects of the system.

FIG. 4D is a block diagram of alternative embodiment of a gas and liquid flow regulation system for cell culture, with a focus on gas-based and liquid-based sensors within the system.

FIG. 4E is a block diagram of alternative embodiment of a gas and liquid flow regulation system for cell culture, with a focus on gas and liquid pumps disposed within the gas and liquid flow paths.

FIG. 4F is a block diagram of alternative embodiment of a gas and liquid flow regulation system for cell culture disposed within cell culture incubator, a temperature controlled portion of the system.

FIG. 5A is schematic diagram of an alternative embodiment of a gas and liquid flow regulation system for cell culture, with a focus on an embodiment of a cell culture compartment that includes a gas sparging element within the liquid cell culture medium.

FIG. 5B is an alternative embodiment of a gas and liquid flow regulation system for cell culture, with a focus on an embodiment of a cell culture compartment that has incoming gas flowing into a gas headspace.

FIG. 5C is an alternative embodiment of a cell culture vessel as a flask stirred by a magnetic stirring bar.

FIG. 5D is an alternative embodiment of a cell culture vessel as gas-permeable cell culture bag on a platform rocker.

FIG. 5E is an alternative embodiment of a gas and liquid flow regulation system for cell culture that includes multiple cell culture vessels.

FIG. 6 is a flow diagram of a method of regulating gas flow and liquid flow within a cell culture instrument.

FIG. 7 is a flow diagram of a method of expanding a cell population within a cell culture instrument.

### DETAILED DESCRIPTION

### Overview and Terminology

It is becoming increasingly appreciated that the phenotypic expression by cells of clinical interest is malleable, and can be affected by culture conditions, such as the inclusion of growth factors, cytokines, and other biologically-active agents in the cell culture medium. Other types of environmental conditions that can have significant effects on the phenotype of cell populations in culture include oxygen level and pressure, both individually and in combination. Accordingly, there is a need in the market for cell culture systems that meet the standards for clinical manufacturing, can control environmental variables such as oxygen level and pressure, and can scale up to deliver multiple liter volumes of cultured cells with a desired phenotypic profile.

Embodiments of the technology are broadly directed to a closed cell culture environmental system having regulated and feedback-controlled gas and liquid flow paths. Embodiments of the technology may be directed to the design of experiments directed toward an understanding of the effects of hyperbaric or hypoxic conditions on a cell population, a human cancer cell population in particular. Embodiments of the technology may also be directed to the scale up and optimization of cell culture technologies for the manufacturing of cell populations for use in medical therapies. One example is for the manufacture of chimeric antigen receptor expressing T cells (CAR-T) for use in immunotherapy regimens.

### Gas and Liquid Flow Regulation

Gas and liquid flow paths both transit through a cell culture vessel included in the closed cell culture environmental system. A system being closed refers to it being sealed against any external environmental liquid or gas, and thus, if sterile at the outset, it remains sterile. Feedback control of gas and liquid flow paths includes sensor-based data from both paths, these data being received by a controller, which, in turn, controls gas and liquid movement through their respective flow paths within the cell culture environmental system. Prominent within the sensory feedback data are oxygen levels and total atmospheric gas pressure levels, in both the gas flow path and the liquid flow path.

Sensor-based data report the composition and pressures of liquid and gas within their respective flow paths. Those data reflect the liquid and gas inputs into the system, but are also reflective of activity of the cells within the cell culture vessel, as described further below. The purpose of collecting sensory data and directing into controlling liquid and gas flow is to provide real-time monitoring of cells within the culture vessel, as well as to adjust liquid and gas flows in order to maintain cell density and cell quality within desired specifications. Real-time monitoring allows for real-time, dynamic control of liquid or gas flow.

The closed cell culture environmental system has multiple controlled inputs into both the liquid and gas flow paths. For example, liquid movement through the system is controlled. Liquid input may include a complete liquid cell culture medium as well as liquid cell culture medium component solutions which may, for example, be liquid subsets of the total medium composition, typically at a relatively high concentration. Component solutions can also convey bioactive agents, such as growth factors or cytokines.

Liquid cell culture medium may also include dissolved gases, such as nitrogen, oxygen, and carbon dioxide. The levels of dissolved gases are a function of the composition of atmospheric gases and more particularly, to the partial pressure levels of individual gases with which the cell culture medium interfaces.

Gas input to the system is also controlled, typically by way of atmospheric gas injected into the system and by pneumatic flow control mechanisms, such as pumps, fans, and valves. Gas input immediately affects the composition and pressure of gases within the system, but gas input, by way of a gas-liquid interface, also affects the composition of dissolved gas within the liquid cell culture medium circulating through the liquid flow path. In one example, liquid cell culture medium is equilibrated with atmospheric gases to achieve a desired dissolved gas profile prior to that medium being moved into the cell culture vessel.

A cell population being cultured in the cell culture vessel can affect the composition of the liquid cell culture medium circulating within the liquid flow paths. These effects can be used to monitor the status of the cell population by way of relevant sensors, such as sensors for glucose, lactate, pH, oxidation/reduction potential, or any useful analyte. Upon receipt of these data, a system controller can adjust gas or liquid flow parameters, as for example, the input flow of nitrogen, or the input flow complete medium or of medium component solutions. The purpose of such flow adjustments is to drive the cell population toward a desired state of metabolism or phenotypic expression, as described further below.

Gas composition within the system may also be affected by cellular metabolic activity, such as the levels of atmospheric or dissolved oxygen. Upon receipt of these data from sensors positioned either in gas or liquid flow paths, a controller can appropriately adjust gas or liquid flow parameters. The purpose of such adjustments is to drive the cell population toward a desired state of metabolism or phenotypic expression.

Other relevant outputs and responses to such input may include temperature of either gas or fluid. Some embodiments of the closed cell culture environmental system may include vibrator units that impart vibrational energy into the cell culture vessel. Vibration may be applied to keeping cells in suspension or stirring them within the cell culture vessel. Vibration may also have advantageous effects on phenotypic expression of cells in culture. In such embodiments, movement sensors can track vibration frequency and amplitude and provide data to a controller, which can then adjust the level of activity of the vibrator.

Some embodiments of the closed cell culture environmental system may include imaging or impedance monitors that provide visual or electrical profiling of the cell population. These profiling capabilities can reflect basic cell culture parameters such as cell viability, cell size, cell density, or more particular cell features that are informative of cell phenotype.

### Cell Phenotype and Cell Culture Performance

Parameters of cell quality and cell culture performance include measures of cell density and metabolism, but also the expression of desirable phenotypic features of the cell population in the culture vessel. The composition and pressures of gas and liquid that support the expression of particular phenotypic features can be cell-type specific, and are determined experimentally.

Phenotype can be observed in any manner, for example, by way of microscopy, or impedance, or any method of observing cells in culture. One example of phenotype relates to potency level within a range extending from multipotency, as in stem cells, to a fully and terminally differentiated cell type. More broadly, phenotype may be captured in the form of and observed aspect or parameter of cell culture performance. At a molecular level, phenotype can manifest as difference in messenger RNA expression of the cellular genome, or rates of protein transcription from expressed RNA.

Cell culture performance parameters that can be related to phenotype or to phenotype as it manifests under various environmental conditions, merely by way of example, may include growth rate, cell death rate, achievable cell density, rate of production of a cell product (natural or transfection-based), cell morphology, cell dimension, cell adherent properties, cell electrical properties, cell metabolic activity, cell migratory behavior, cell activation state, biomarker demonstration, amenability or resistance to transfection, vulnerability or resistance to infection, responsiveness or resistance to a bioactive agent, or any other observable aspect of cell phenotype or function.

### Control System and Workflow

Some embodiments of the closed cell culture environmental system are configured to have multiple cell culture vessels under the control of a system controller. The system controller may be singular, or it may be arranged as an integrated control system wherein a master controller runs multiple dependent controllers. The specific control responsibilities of system having a master controller and multiple slave controllers may divided in multiple ways.

Some embodiments of the closed cell culture environmental system are configurable into a larger automated workflow that includes cell isolation and transduction steps, or steps related to quality control or regulatory compliance.

### Terminology: Gas Pressure, Oxygen, Cell Density, a Well-Mixed Environment

Embodiments of the closed cell culture environmental system are broadly directed to creating hyperbaric and hypoxic atmospheric conditions in a cell culture environment. In some instances, a hyperbaric and hypoxic atmospheric condition is referred to as a “tumor microenvironment” (TME) because tumors in the natural environment of the body are typically hyperbaric and hypoxic compared to other compartments in the body, as compared to a typical atmospheric condition within a conventional CO2 incubator.

Hyperbaric refers to a total atmospheric gas that is higher than that of ambient gas pressure. Gas pressure can be measured using a number of different terms, including bars, atmospheres (ATM), megapascals (MPa), a column of mercury (mmHg), a column of water (WC inch), gas pounds/square inch (PSI), and gas pounds/square inch gauge (PSIG). PSIG refers to the pounds/square inch above ambient gas pressure, however it is also common to simply use PSI when it is understood that it also refers to gas pressure above ambient level. This is the convention in many instruments, and is also used herein.

Hypoxic or hypoxia refer generally to a condition of low oxygen level; it is a term that is comparative to what may be considered a normal oxygen level, as for example, the oxygen level of normal ambient atmosphere, or a well-mixed liquid at a soluble gas equilibrium with a normal ambient atmosphere.

When referring to oxygen in gas phase, hypoxic or hypoxia refers to an oxygen level that is lower than that in the ambient atmosphere. In the earth's atmosphere, oxygen is present at approximately 21%; this is a relative percent value, i.e., 21% of the molecules in a given atmospheric gas volume are oxygen molecules.

A separate term, partial pressure, refers to the oxygen level in absolute terms, i.e., the number of molecules in a given volume without regard to the level of any other gas molecules in the same volume. Using oxygen as an example, inasmuch as the partial pressure of oxygen is independent of the presence of other gas molecules, the partial pressure of oxygen within a given volume is also independent of the total gas pressure within the same volume.

Although many instruments report oxygen level as a % value, that value is based on an actual sensing of oxygen partial pressure. Further relevant is that the equilibrium-driven force that distributes atmospheric oxygen into liquid is the oxygen partial pressure. For these various foregoing considerations, it should be understood that for this patent application, partial pressure is what is meant generally by any expression of oxygen level.

An oxygen level used in a method disclosed herein can be, for example, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, or about 25% oxygen in the incubator.

A pressure used in a method disclosed herein can be about 0 PSI, about 0.1 PSI, about 0.15 PSI about 0.2 PSI, about 0.25 PSI, about 0.3 PSI, about 0.35 PSI, about 0.4 PSI, about 0.45 PSI, 0.5 PSI, about 0.55 PSI, about 0.6 PSI, about 0.65 PSI, about 0.7 PSI, about 0.75 PSI, about 0.8 PSI, about 0.85 PSI, about 0.9 PSI, about 0.95 PSI, about 1 PSI, about 1.1 PSI, about 1.2 PSI, about 1.3 PSI, about 1.4 PSI, about 1.5 PSI, about 1.6 PSI, about 1.7 PSI, about 1.8 PSIG, about 1.9 PSI, about 2 PSI, about 2.1 PSI, about 2.2 PSI, about 2.3 PSI, about 2.4 PSI, about 2.5 PSI, about 2.6 PSI, about 2.7 PSI, about 2.8 PSI, about 2.9 PSI, about 3 PSI, about 3.5 PSI, about 4 PSI, about 4.5 PSI, about 5 PSI, about 6 PSI, about 7 PSI, about 8 PSI, about 9 PSI, or about 10 PSI. A pressure used in a method disclosed herein can be an above atmospheric pressure value. A pressure used in a method disclosed herein can be a hyperbaric pressure.

A pressure used in a method disclosed herein can be a PSI gauge (PSIG) reading of, for example, about 0.5 PSIG, about 0.6 PSIG, about 0.7 PSIG, about 0.8 PSIG, about 0.9 PSIG, about 1 PSIG, about 1.1 PSIG, about 1.2 PSIG, about 1.3 PSIG, about 1.4 PSIG, about 1.5 PSIG, about 1.6 PSIG, about 1.7 PSIG, about 1.8 PSIG, about 1.9 PSIG, about 2 PSIG, about 2.5 PSIG, about 3 PSIG, about 3.5 PSIG, about 4 PSIG, about 4.5 PSIG, about 5 PSIG, about 6 PSIG, about 7 PSIG, about 8 PSIG, about 9 PSIG, about 10 PSIG, about 15 PSIG, about 20 PSIG, about 25 PSIG, about 30 PSIG, about 35 PSIG, about 40 PSIG, about 45 PSIG, about 50 PSIG, or about 55 PSIG.

A pressure used in a method disclosed herein can be, for example, about 3.45 kPa, about 4.14 kPa, about 4.83 kPa, about 5.52 kPa, about 6.21 kPa, about 6.89 kPa, about 7.58 kPa, about 8.27 kPa, about 8.96 kPa, about 9.65 kPa, about 10.3 kPa, about 11 kPa, about 11.7 kPa, about 12.4 kPa, about 13.1 kPa, about 13.8 kPa, about 17.2 kPa, about 20.7 kPa, about 24.1 kPa, about 27.6 kPa, about 31 kPa, about 34.4 kPa, about 41.4 kPa, about 48.3 kPa, about 55.2 kPa, about 62.1 kPa, about 68.9 kPa, about 103 kPa, about 138 kPa, about 172 kPa, about 207 kPa, about 241 kPa, about 276 kPa, about 310 kPa, about 345 kPa, or about 379 kPa.

### Cell Density Terminology

As used herein, cell density generally relates an absolute number of cells per unit volume of cell culture medium. In particular embodiments the cell culture density relates to an absolute number of viable cells per unit volume of cell culture medium. In some embodiments, a cell culture density value relies on a surrogate reflection of an absolute number of cells per unit volume of cell culture medium; for example, the ATP concentration of a cell culture lysate can serve as cell density surrogate. In some embodiments, cell density relates to the sum total cellular volume per unit cell culture medium volume. In some embodiments, cell density relates to the sum total cellular mass per unit cell culture medium volume.

Cell density is a process parameter for monitoring and controlling a cell culture process. In some instances, there may be relatively little control exerted over cell density, for example, a cell culture may be simply allowed, passively to go to a maximum level. In other instances, a particular cell density may be desired. In this latter case, one form of controlling the cell density is by controlling the volumetric perfusion rate, in which case the cell density serves as a feedback control on either the composition of gas being fed into a medium gassing chamber (as described below) or on the rate of cell culture medium flowing into and out of a cell culture container, such as a cell culture bag (as described below).

### Mixing Medium and Distributing Cells in a Volume of Cell Culture Medium

It is advantageous for a cell culture that cells be substantially uniformly distributed within their liquid medium, as this gives individual cells within the cultured population to have substantially equal access to nutrients in the liquid cell culture medium. It is advantageous for a cell culture that culture medium composition to be substantially uniform throughout the cell culture vessel as this gives individual cells within the population to have substantially equal access to the same level of nutrients in the liquid cell culture medium. It is advantageous for a cell culture's dissolved gases be uniformly distributed throughout the liquid cell culture medium volume, as this provides that individual cells within the cultured cell population to be exposed to the same local composition of dissolved gases. Accordingly, embodiments of the technology provided herein include approaches to mixing or agitation of cell culture medium, and the cells contained therein, within a cell culture vessel.

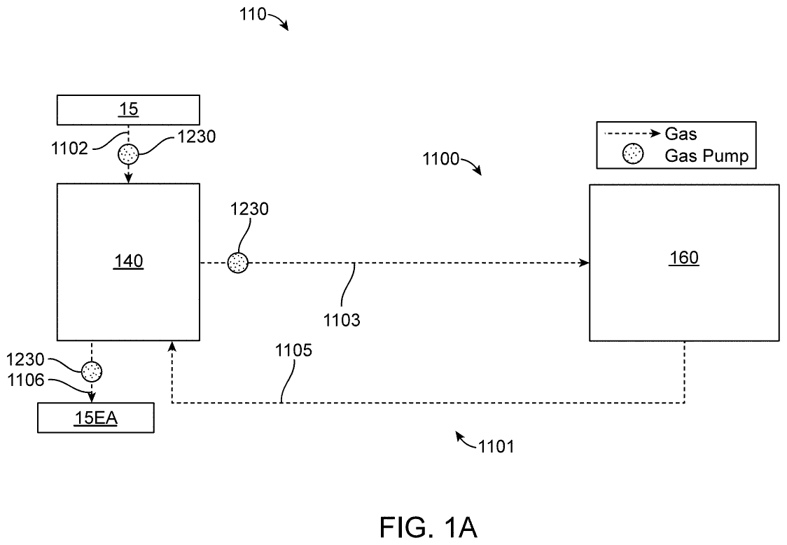

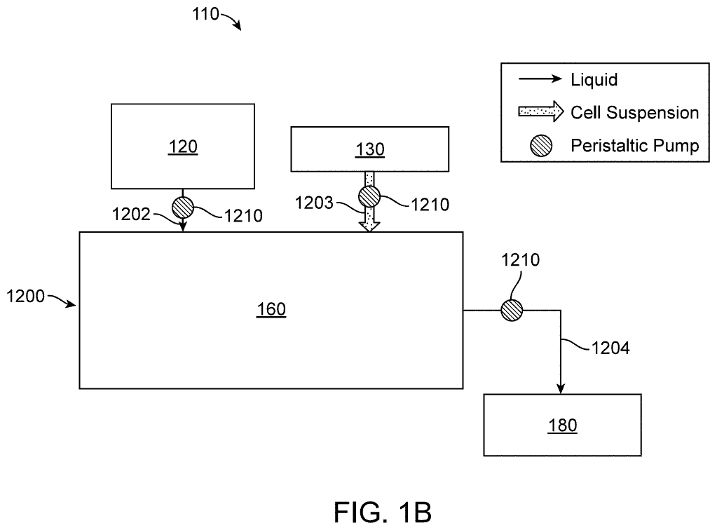

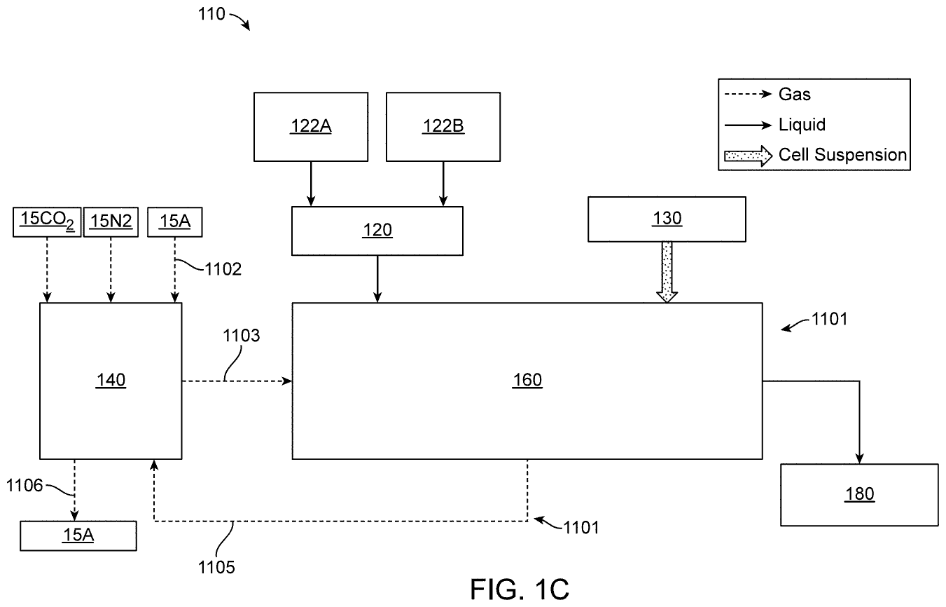

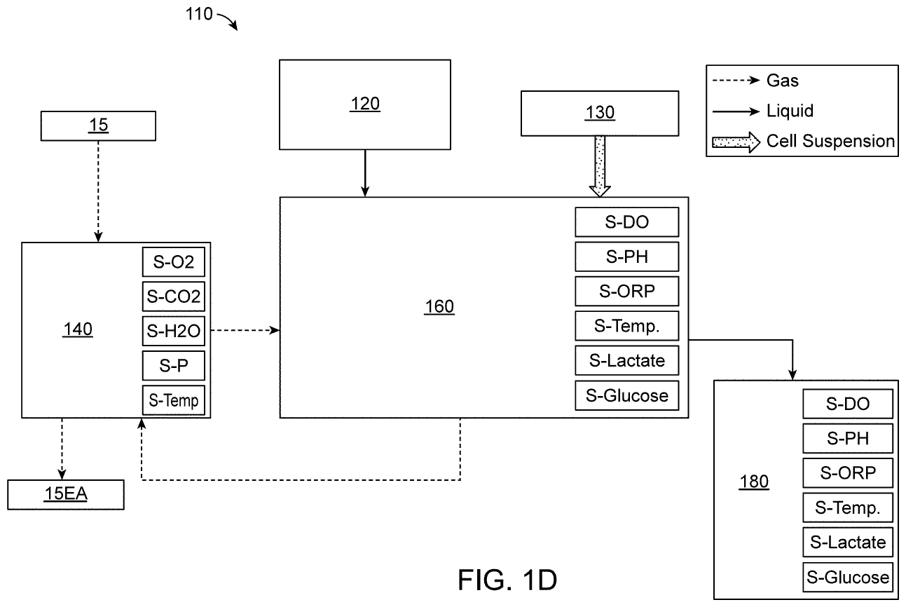

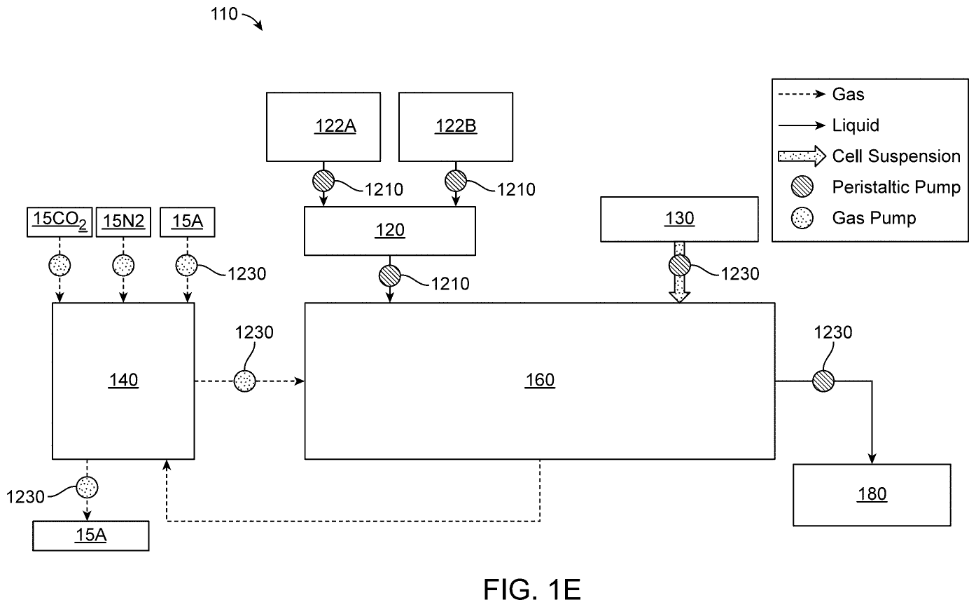

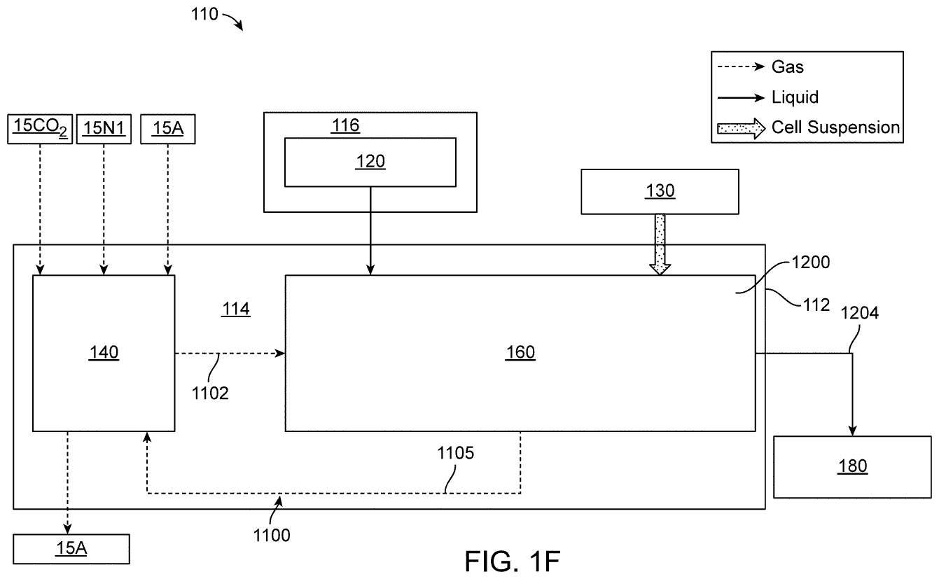

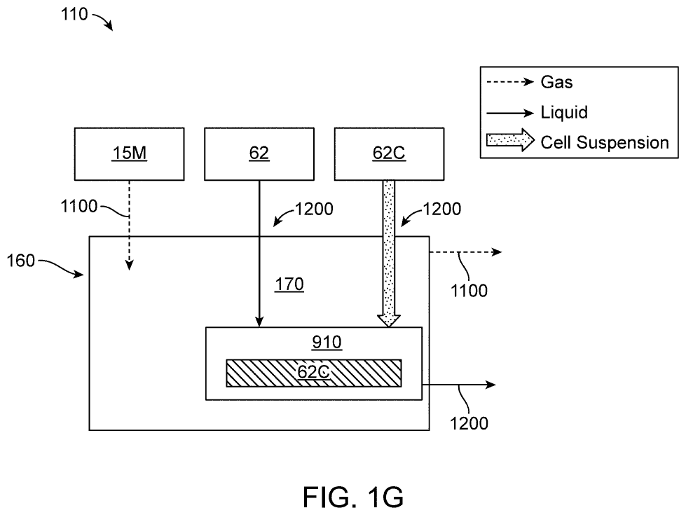

FIGS. 1A-1G includes block diagrams and a schematic diagram of an embodiment and aspects of an embodiment of a gas and liquid flow regulation system 110, collectively showing separate but intertwined paths for gas flow (driven by pneumatic pressure) and liquid flow (driven by hydraulic pressure). In brief, FIG. 1A is a block diagram that focuses on the gas flow aspect of the system. FIG. 1B is a block diagram that focuses on the liquid flow aspect of the system. FIG. 1C is a block diagram that shows the combined gas flow (as in FIG. 1A) and liquid flow aspects (as in FIG. 1B) of the system. FIG. 1D is a block diagram that focuses on gas-based and liquid-based sensors within the system. FIG. 1E is a block diagram that focuses on gas and liquid pumps disposed within the gas and liquid flow paths. FIG. 1F is a block diagram that shows of an embodiment of a gas and liquid flow regulation system for cell culture as disposed within a cell culture incubator, a temperature controlled portion of the system. FIG. 1G is a schematic diagram of a cell culture compartment embodiment of a gas and liquid flow regulation system for cell culture as disposed within cell culture incubator, a temperature controlled portion of the system.

FIGS. 1A-1C provide views of a gas and liquid flow regulation system 110 for cell culture at a basic level, with a focus on gas and liquid flow paths through component containers or chambers within the system. Gas containers or chambers include gas sources 115, a pressurizable gas mixing chamber 140, and a cell culture compartment 160. These various components are connected by segments of a gas flow path, as defined by an originating source or container, and a receiving container or vent.

Turning now to gas flow path 1100 and its segments and container portions per FIG. 1A, and FIGS. 1C-1F. Gas flow path 1100 includes the various gas containers, such as gas sources 115, pressurizable gas mixing chamber 140, and cell culture compartment 160, as well as gas flow path segments, as described below.

Gas from one or more gas sources 115 is injected into pressurizable gas mixing chamber 140 by way of gas flow path segment 1102. Mixed pressurized gas from pressurizable gas mixing chamber 140 flows into cell culture compartment 160 by way of flow path segment 1103. Pressurized gas from cell culture compartment 160 flows back to pressurizable gas mixing chamber 140 by way of gas flow path segment 1105. Pressurized gas within pressurizable gas mixing chamber 140 can be released into the ambient environment by way of vent 1106.

Each gas flow path segment (1102, 1103, 1105, 1106) includes an exit port from its respective originating container and an entry port into the receiving container. The flow rate of gas through flow paths is typically regulated by gas pumps 1230 within the paths, as shown. Gas flow path segments, collectively and typically, form a circular loop 1101 that is closed to gas entry or exit other than controlled entry from gas source 115 and exit through vent 1106. The total gas pressure within this loop, including the various containers within the loop (pressurizable gas mixing chamber 140 and cell culture compartment 160) has a transient dynamic, but overall remains substantially the equivalent throughout. Gas pressure within components of circular loop 1101 is generated by a pneumatic pump 1230, positioned between pressurizable gas mixing chamber 140 and cell culture compartment 160. Gas flow within gas flow system 1100, and particularly within circular loop portion 1101, is typically continuous, during operation of gas and liquid flow regulation system 110.

Pressure within pressurizable gas mixing chamber 140 is contributed to upstream by incoming gas from gas source 115, which maybe compressed gas within the gas source or may be alternatively pushed by a pump or fan. Gas flowing out of pressurizable gas mixing chamber 140 may be further pushed downstream by a gas pump 1230, as also shown in FIG. 1E, which focuses on gas and liquid pumps, and as described below.

Turning next to the liquid flow path 1200 portion of gas and liquid flow regulation system 110 per FIGS. 1B-1C. Embodiments of liquid flow path 1200 include containers or compartments linked by flow path segments. Fresh liquid cell culture medium is included within cell culture medium source container 120; cell inoculum source container 130 holds fresh cell culture medium with cells at an inoculum density; cell culture compartment 160 hosts a volume of cell culture medium with cells; and downstream culture container 180 may assume various functions, including waste or sampling. Cell-free culture medium from medium source container 120 flows into cell culture compartment 160 by way of liquid flow path segment 1202. After liquid cell culture medium has been exposed to gas-phase gas, as it is within cell culture compartment 160, the dissolved gas in the liquid cell culture medium becomes equilibrated with the gas-phase gas, according to solubility of each individual gas in the liquid medium, and according to the partial pressure of the individual gas in the gas phase gas.

As noted, liquid cell culture medium from culture medium source container flows into cell culture compartment 160 by way of liquid flow path segment 1202. Cell-containing culture medium from cell culture inoculum container 130 flows into cell culture compartment 160 by way of liquid flow path segment 1203. Liquid cell culture medium (either cell-free, partially cell-free, or cell-containing) flows from cell culture compartment 160 into downstream culture container 180 by way of liquid flow path segment 1204. Liquid cell culture medium flow within the various liquid flow path segments and containers is driven by hydraulic pressure, typically generated by peristaltic pumps, as described further below. As liquid arrives in any container, it passes through an entry port of that container. As liquid leaves any container, it passes through an exit port of that container.

FIG. 1C shows the shows the combined gas flow (as in FIG. 1A) and liquid flow aspects (as in FIG. 1B) of system 110, and adds some further detail. Gas source 115 is typically manifested in the form individual gas sources for carbon dioxide 115CO₂, nitrogen 115N₂, and ambient air 115A. Gas sources for carbon dioxide 115CO₂ and nitrogen 115N₂ are typically compressed gas cylinders; gas flows from them into pressurizable gas mixing chamber 140 as controlled by valves into the pressurizable gas mixing chamber. Ambient air from the external environment is filtered, and then blown into by a pump or fan. FIG. 1C also shows an arrangement whereby culture medium container 120 is fed by two medium component containers 122A and 122B, which contain distinct medium components.

Gas flow path 1100 includes circular flow path portion 1101, which includes a first gas flow path segment 1103 from the pressurizable gas-mixing chamber to the cell culture compartment and a second gas flow path segment 1105 from cell culture compartment back to the pressurizable gas-mixing chamber, wherein the gas flow system is adaptable to provide a hyperbaric and hypoxic atmospheric condition within the cell culture compartment. Gas flow path 1100 further includes pressurizable gas mixing chamber 140 and cell culture compartment 160.

The circular portion 1101 of the gas flow system is closed to entry of gas from an environment external to external gas, and the hyperbaric and hypoxic atmospheric condition, as generated in pressurized gas mixing chamber 140, is substantially uniform throughout. At least in part, the hyperbaric atmospheric condition within the gas flow system is supported by an application of pneumatic pressure from the multiple gas sources. Further, the circular portion of the gas flow system is configured for continuous circular gas flow when the gas flow system is in operation.

Although embodiments of a gas flow regulation system, as described herein, are broadly directed to creating a hypoxic environment in gas phase gas and in the dissolved gas of cell culture medium, the described system could be modified to create a hyperoxic (high oxygen) environment. The modifications would include the input of oxygen into the system directly from an oxygen source, rather than the oxygen within ambient air, which is the oxygen source in described in various embodiments herein.

FIG. 1D shows an embodiment of a gas and liquid flow regulation system 110 for cell culture in accord with FIG. 1C, with a particular focus on sensors disposed within the gas flow path 1100 and liquid flow path 1200. Sensors that can be deployed within gas spaces of the system may include any one or more of an oxygen sensor S-O2, a carbon dioxide sensor S-CO2, a water or humidity sensor S-H2 0, a pressure sensor S-P. Gas spaces within gas flow regulation system 100 include containers, such as gas mixing chamber 140 and cell culture compartment 160.

Sensors that can be deployed within liquid spaces of the system may include any one or more of a dissolved oxygen sensor S-DO, a carbon dioxide sensor S-CO2, a glucose sensor S-glucose, a lactate sensor S-lactate, a pH sensor S-PH, an oxidation-reduction potential (ORP) sensor S-ORP, and a temperature sensor S-Temp. Other sensors may be included, such as, merely by way of example, for particular amino acids included in the cell culture medium, or for associated metabolites thereof. Liquid spaces within liquid flow regulation system 1200 include liquid cell culture medium source container 120, cell inoculum source container 130, cell culture compartment 160, and downstream collection container 180. Liquid flow path segments between containers include flow path 1102 from gas source 115 to pressurizable gas mixing chamber 140, flow path 1103 from pressurizable gas mixing chamber 140 to cell culture compartment 160, and flow path 1105 from cell culture compartment 160 back to pressurizable gas mixing chamber 140.

FIG. 1E shows an embodiment of a gas and liquid flow regulation system 110 for cell culture in accord with FIG. 1C, with a particular focus on and liquid pumps 1210 and gas pumps 1230 disposed within the liquid and gas flow paths, respectively. Gas pumps move gas through the gas flow by way of atmospheric or pneumatic pressure. Gas pumps can be of any suitable type, and as herein, gas pressure applied by such mechanisms as fans or compressed gas containers may also be referred to as pneumatic pumps. Liquid pumps move liquid cell culture medium through the gas flow path by way hydraulic pressure. In typical embodiments, liquid pumps are exemplified as peristaltic pumps, but any suitable hydraulic movement mechanism is included as a hydraulic pump.

FIG. 1F shows an embodiment of a gas and liquid flow regulation system 110 for cell culture disposed within cell culture instrument 112. In this embodiment, cell culture compartment 160 is positioned inside cell culture instrument 112. Disposed within cell culture instrument 112 is a temperature-controlled compartment 114, and disposed within temperature-controlled compartment 114 is cell culture compartment 160. Controlling the temperature of cells in cell culture compartment 160, typically at or around 37° C., is essential for consistent cell culture runs.

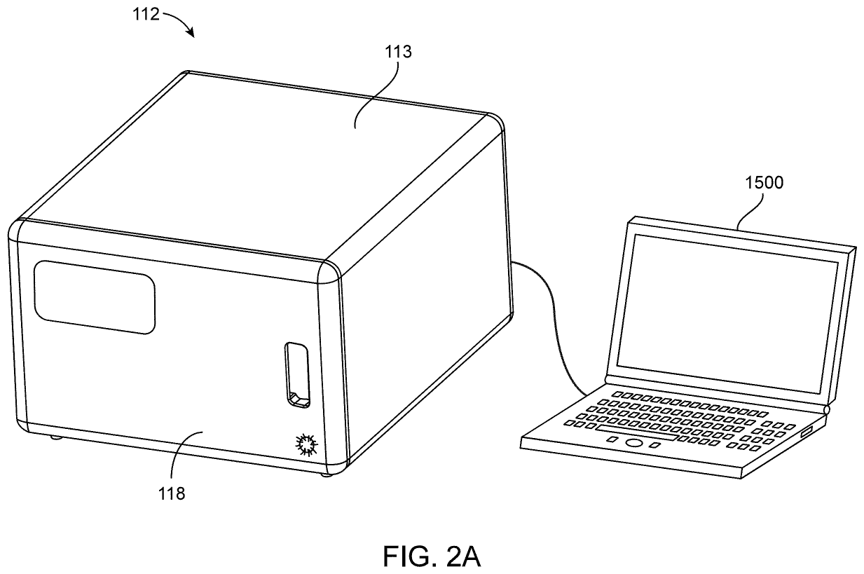

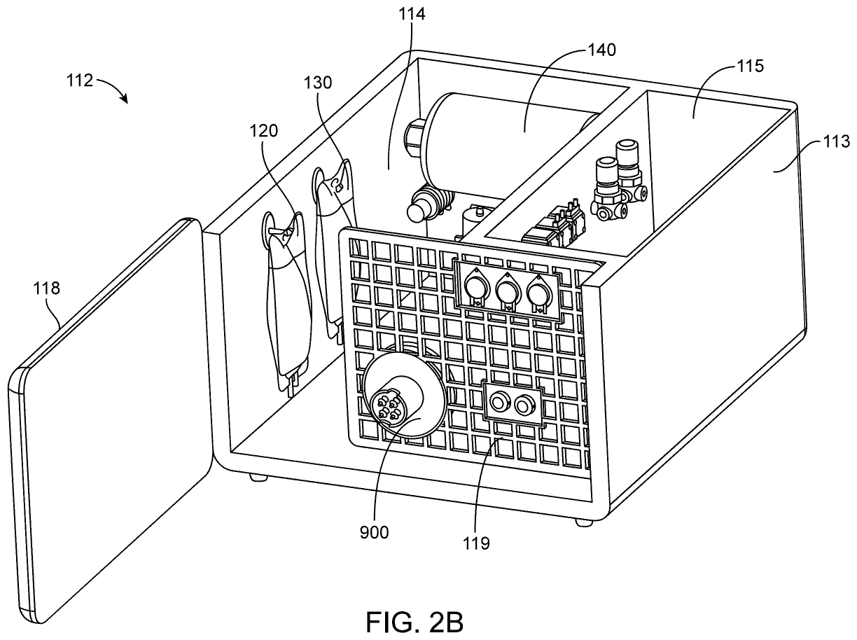

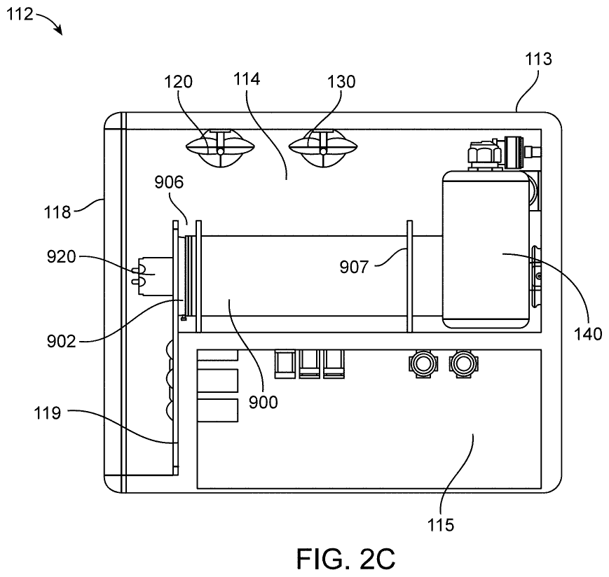

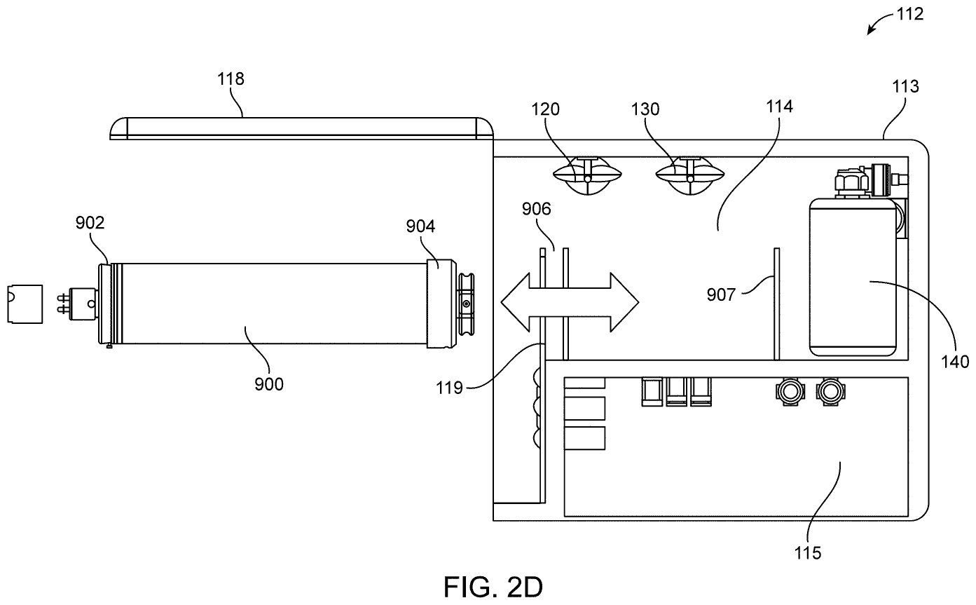

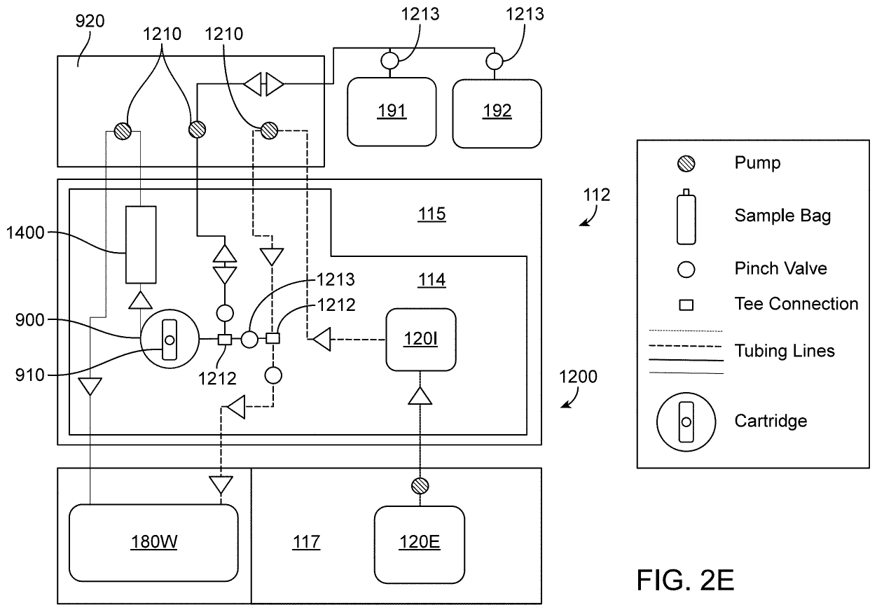

Components of system 110 that may be positioned external to the incubator 112 include pressurizable gas mixing chamber 140, culture medium source container 120 (which may be refrigerated) and cell inoculum source container 130. In some embodiments, there may be an advantage to pressurizable gas mixing chamber 140 being external to cell culture instrument 112 in that can allow that chamber to serve more than one incubator. Containers such as gas sources 115 and downstream culture container 180 are sensibly positioned outside incubator 112. An electronics-hosting compartment 115 is not shown in FIG. 1F (instead, see FIGS. 2B-2D and associated description further below). Electronics-hosting compartment 115 is typically not temperature-controlled, in contrast to temperature-controlled compartment 114.

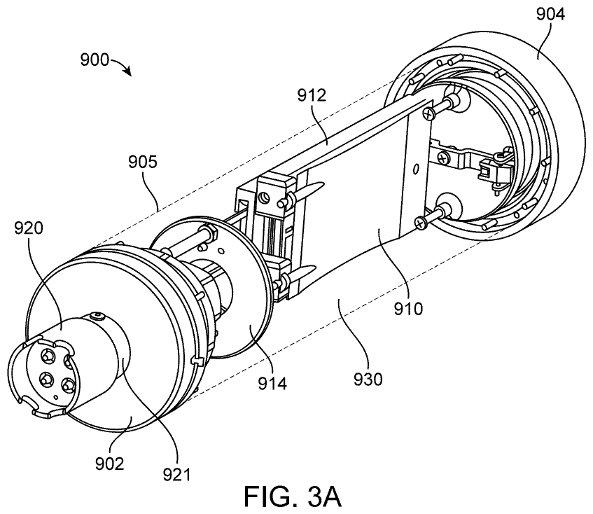

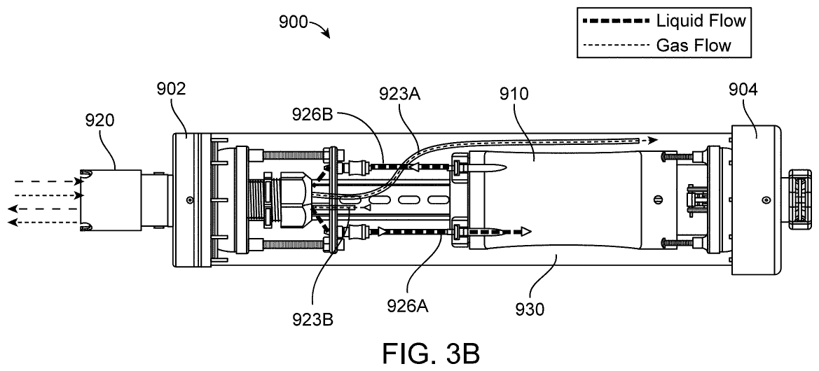

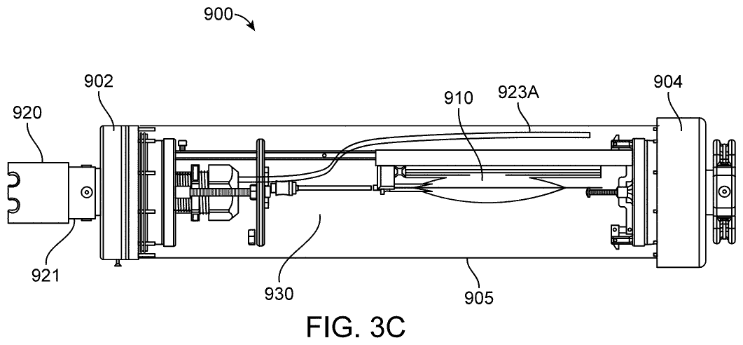

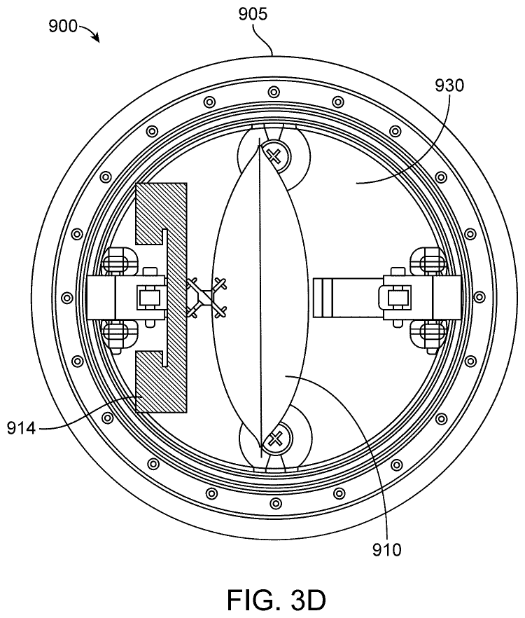

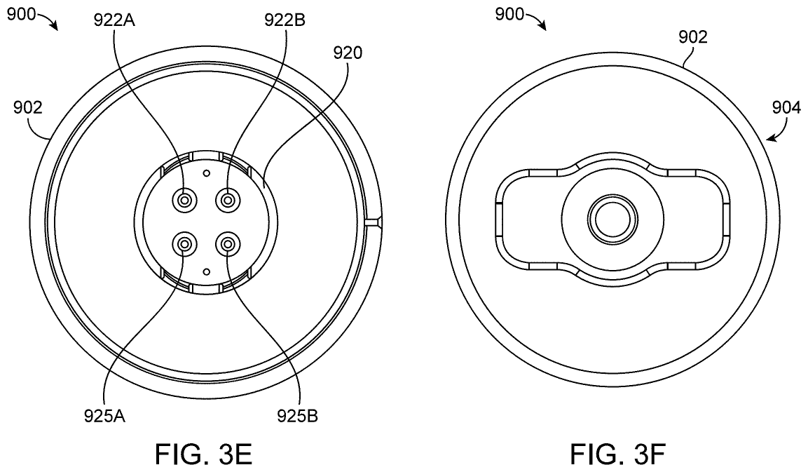

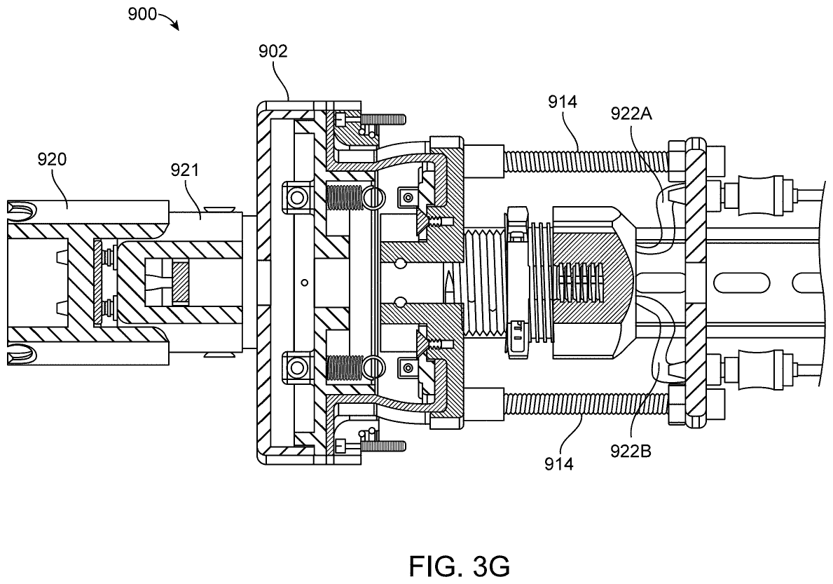

FIG. 1G is a schematic diagram of an embodiment of a gas and liquid flow regulation system 110 for cell culture, with a focus on an embodiment of a cell culture compartment 160 that has a gas-permeable cell culture bag disposed within a gas space 170 (see FIGS. 2A-2E and FIGS. 3A-3G). In this embodiment, gas-phase gas of the gas flow regulation system and the liquid of liquid flow regulation system interface at the surface of cell culture bag 910.

Mixed, pressurized gas 15 (contributed to by one or more sources) flows into gas space 170 of cell culture compartment 160. Fresh liquid cell culture medium 62 flows into cell culture bag 910 (an embodiment of a cell culture vessel) as needed. Liquid cell medium with an inoculum of cells 62C also flows into cell culture bag 910 as needed. Liquid medium with cells 62C can flow out of cell culture bag 910 into any of various downstream containers. Gas from within gas space 170 can flow out of cell culture bag 910 to return to pressurizable gas mixing chamber 140, as seen in and as described above in context of FIGS. 1A and 1C-1F, thereby forming a circular portion 1101 of gas flow system 1100.

The foregoing description of a cell culture instrument and gas and liquid flow components contained therein, and the rationale for the described and depicted configurations notwithstanding, other arrangements of gas and liquid flow components may be advantageous for various circumstances. All such alternative configurations are within the scope of the disclosed technology.

FIGS. 2A-2E are views of a cell culture instrument 112 that houses a gas flow system and a liquid flow system disposed within a cell culture compartment that is operably connected to both the gas flow system and the liquid flow system. These various components are detailed in FIGS. 1A-1G, that depict a gas flow system 1100 and a liquid flow system 1200, and cell culture compartment 160. Cell culture instrument 112, in typical embodiments, is configured to stackable, so that two or more instruments can be stacked on top of each other, and accommodated within the same footprint in a laboratory of manufacturing facility.

FIGS. 2A-2E are views of a cell culture instrument 112 that houses a gas flow system 1100 and a liquid flow system 1200 disposed within a cell culture compartment 160 that is operably connected to both the gas flow system and the liquid flow system.

FIGS. 2A-2E are views of an embodiment of the technology as housed within cell culture instrument 112, now described in more detail. FIG. 2A is a perspective view of cell culture instrument 112 that houses a gas and liquid flow regulation system 110. Depicted also in FIG. 2A are a housing 113 that supports the instrument, a front door 118 that provides access into the interior of the instrument, a front support panel 119, which supports a cell culture cartridge and entry and sites for liquids, and a controller 1500 operably connected to cell culture instrument 112.

FIG. 2B is a perspective view of cell culture instrument 112 that houses gas and liquid flow regulation system 110; front door 118 is open and the top removed to provide a view into the interior of the instrument. Within the interior of the cell culture instrument 112 are two main spaces, a temperature-controlled space 115 and an electronics hosting space 115 that is not temperature-controlled. A front support panel 119 interfaces between the interior of 112 and front door 118. The front end of a cell culture cartridge 900 projects through the front support panel and into a space that can accommodate tubing that extends into the interior of temperature-controlled space 114. cell culture cartridge 900 is described below, and depicted in FIGS. 3A-3F.

FIG. 2C is a top view of cell culture instrument 112 that houses a gas and liquid flow regulation system 110; the top is removed to provide a view into the interior of the instrument. FIG. 2D is a top view of cell culture instrument 112 that houses a gas and liquid flow regulation system; front door 118 is open and the top removed to provide a view into the interior of the instrument; cell culture cartridge 900 is shown outside the instrument, aligned with a receptacle within the instrument. Cell culture cartridge 900 is a particular embodiment of a cell culture compartment 160, as described above and depicted in FIGS. 1A-1G. A receptacle for cell culture cartridge 900 is provided by a combination of an entry port 906 within front support panel 119 and a back end receptacle support 907.

FIG. 2E is a schematic diagram that shows details of a system of liquid flow regulation 190 within cell culture instrument 112, the liquid containers and the paths that guide liquid flow from one container to another. Major compartments of cell culture instrument 112 include a temperature-controlled portion 114, which may be referred to as an incubator, and a non-temperature controlled portion 115, which houses electronic aspects of the instrument. A tube mount 920 is shown as a block, but in actual configuration is mounted at the front end of a cell culture cartridge 920 that provides entry and exit of liquid into the cartridge, and into a cell culture bag 910 contained therein. (Cell culture cartridge 900 and cell culture bag 910 are detailed in FIGS. 3A-3G.)

External to the instrument is a refrigerated compartment 117 to hold fresh cell culture medium or any other liquid component that should be kept refrigerated. Other medium feed or medium collection containers are typically or optionally external to the incubator portion of the cell culture instrument.

As seen in FIG. 2E, liquid flow path begins with a liquid cell culture medium container 120E as source for fresh liquid medium into the flow path. Other cell culture medium containers include an optional medium container 120I disposed within a temperature-controlled space of instrument 112, and a cell culture vessel in the form of a cell culture bag 910, positioned inside a cell culture compartment in the form of cell culture cartridge 900 (cartridge and cell culture bag as shown in FIGS. 3A-3G). An advantage offered by internal medium container 120I is that it warms incoming cell culture medium, and does not disturb the temperature of medium in the cell culture bag 900. However. in some applications that may not be necessary, as for example, when the volume of cell culture medium moving into the cell culture bag is small compared to the standing volume within the bag. As an alternative to the internal medium container, a liquid tubing line may include a coiled section, to increase the rate of heat transfer from the temperature-controlled incubator into the incoming medium.

Further included in the liquid flow system are upstream containers for liquid upstream feeds 191 into cell culture cartridge 900 and downstream liquid collection containers 192 to receive liquid therefrom. Upstream liquid containers 191 may have particular cell culture medium components (e.g., concentrated liquid components, bioactive agents, or viruses for transfective functions) or it may have cells distributed in medium to inoculate the cell culture compartment 900. Downstream liquid containers 192 may include containers for samples of cells or cell-conditioned medium, or cell culture medium or cells to collect as product, or to remove as waste. (Cell-conditioned refers to cell culture medium that has already hosted cells.) A container 1400 or portion of a medium flow line exiting the cell culture compartment 1400 includes an array of liquid based sensors to monitor the state of cell culture medium immediately as it exits the cell culture compartment, prior to be removed as waste into 180W (as below). Liquid-based sensors may also be included elsewhere in the liquid flow path.

The liquid flow path also includes a waste container 180W, typically external to the incubator, which can include input from the cell culture compartment, or for clearing cell culture medium from liquid flow lines that are located prior to entry into the cell culture compartment.

Liquid is moved from one container to another by way of hydraulic pumps (typically peristaltic pumps) 1213 as shown. Directionality of flow is indicated by arrows. In some instances, as indicated by bidirectional arrows, the same liquid flow path or flow path segment (tubing) may be used to flow liquid in either direction. Switching flow direction may include flushing the line with fresh medium. Entry or exit of liquids from containers 191 and 192 may be controlled by pinch valves 1213. T-connections 1212 are positioned at points in the liquid flow path that act as junctions.

FIGS. 3A-3G are views of an embodiment of a cell culture compartment (labeled as 160 in FIGS. 1A-1G) in the particular form of a cylindrical cartridge 900, which is disposed within a cell culture instrument 112 (as shown in FIGS. 2A-2D). It is within cartridge 900 that the liquid cell culture medium within the liquid flow regulation system 1200 and the gas circulating within the gas flow regulation system 1110 interface with each other at the surface of a cell culture bag 910. Noting, in particular, cell culture bag 910 and gas space 930 within cylindrical cartridge 900; it is at the cell culture bag surface where incoming gas-phase gas and dissolved gas in the cell culture medium engage each other, and provide a way to influence the composition of gas dissolved in the cell culture medium by regulating the composition of gas-phase gas to which the medium is exposed.

FIG. 3A is an isometric perspective view of an embodiment of a cell culture cartridge 900 for an embodiment of a gas and liquid flow regulation system for cell culture, showing the flow paths of gas and liquid therethrough. FIG. 3B is a side view of cell culture cartridge 900 for an embodiment of a gas and liquid flow regulation system for cell culture.

FIG. 3C is a top view of cell culture cartridge 900 for an embodiment of a gas and liquid flow regulation system for cell culture. This figure, in particular shows, gas intake line 923A extended to the back of the cartridge to facilitate mixing of gas within the gas space of the cartridge, as gas coming into the gas space 930 arrives in the back of the cartridge, and eventually exits, having mixed with existing gas-phase gas, from the front of the cartridge.

FIG. 3D is a cross-sectional view of a cell culture cartridge 900 for an embodiment of a gas and liquid flow regulation system for cell culture. Tubing scaffold 914 supports cell culture bag 912 in a vertical position, which generally provides an advantageous configuration for the bag, and for distribution of cells within cell culture medium therein.

FIG. 3E is a face view of the exterior of front lid of cell culture cartridge 900 for an embodiment of a gas and liquid flow regulation system for cell culture. Visible within the central portion of the lid are gas entry and exit tubes (922A and 922B, respectively). FIG. 3F is a face view of the exterior of back lid of cell culture cartridge for an embodiment of a gas and liquid flow regulation system for cell culture. Visible within the central portion of the lid are liquid entry and exit tubes (925A and 925B, respectively).

FIG. 3G is a length-wise cross-sectional view of the front end of a cell culture cartridge 900, including, in particular a view of a tube mount connector 920 and a tube stabilizer piece 921. Tube connector 920 and tube stabilizer 921 are attachable and detachable from each other. Tube stabilizer 921 is fixedly attached to cell culture cartridge 900; it functions to stabilize gas and liquid flow tubes, as they enter and exit from the cell culture cartridge. Also shown within cell culture cartridge 900 is a tube scaffold assembly 914, which supports cell culture bag 910 (not shown in this view), and entry gas flow tube 922A and exit gas flow tube 922B.

In some embodiments of the cylindrical cartridge 900 and its receptacle within instrument 112, the cartridge and its receptacle (as described above) are collectively configured to provide agitation of the cell culture bag and its contents, as for example, either by complete rotation or by a partial rotation in the form of a clockwise and counter clockwise rocking.

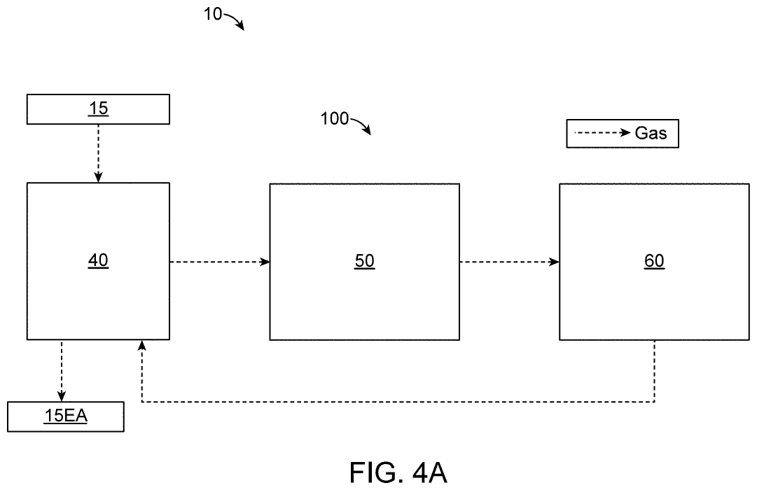

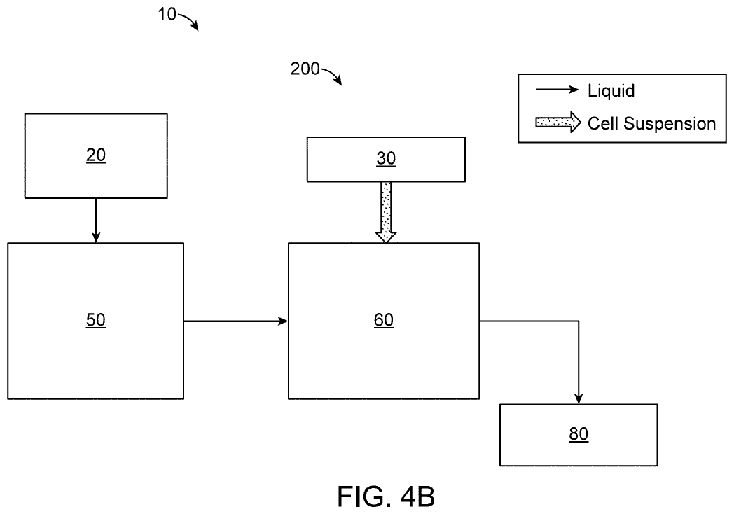

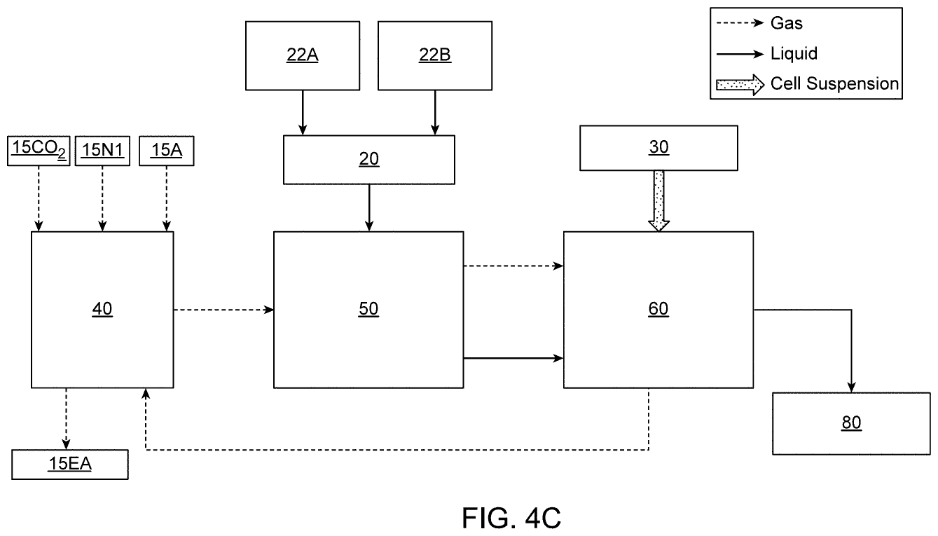

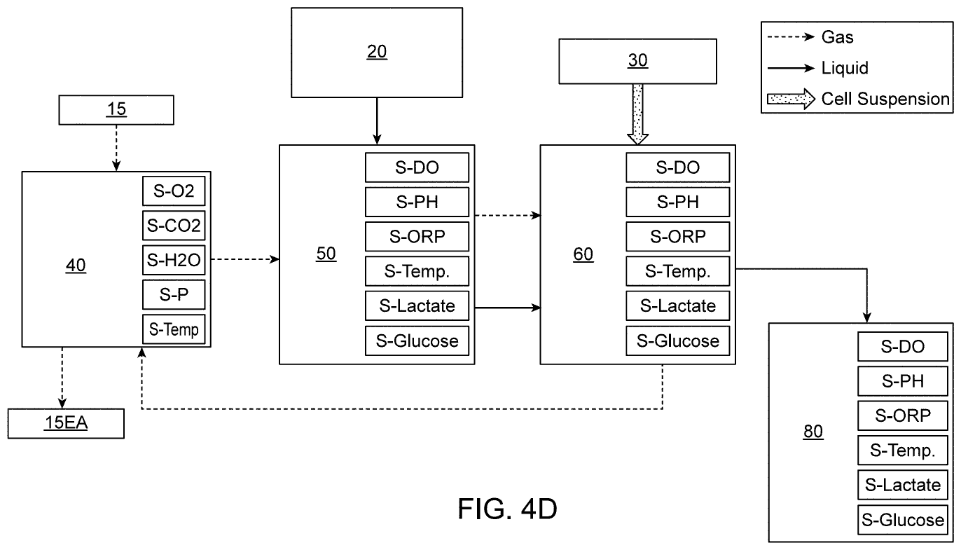

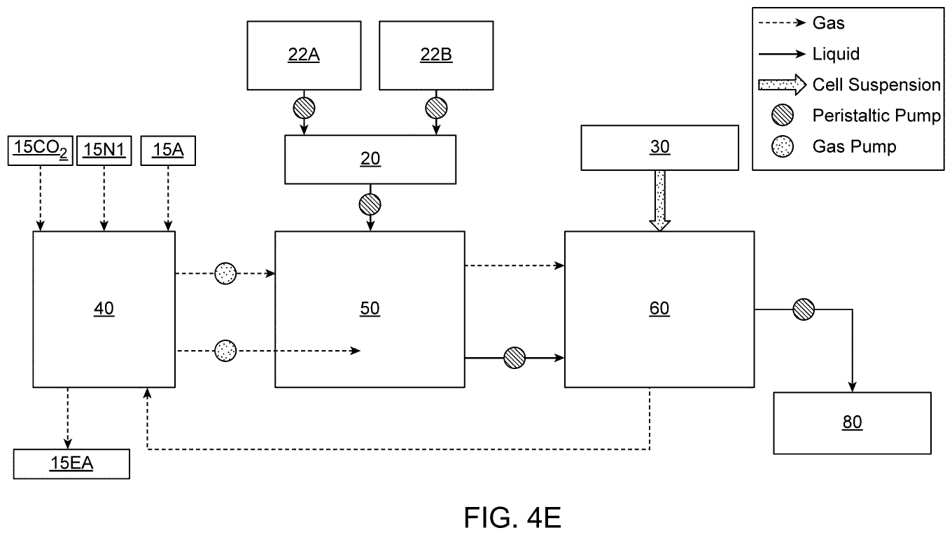

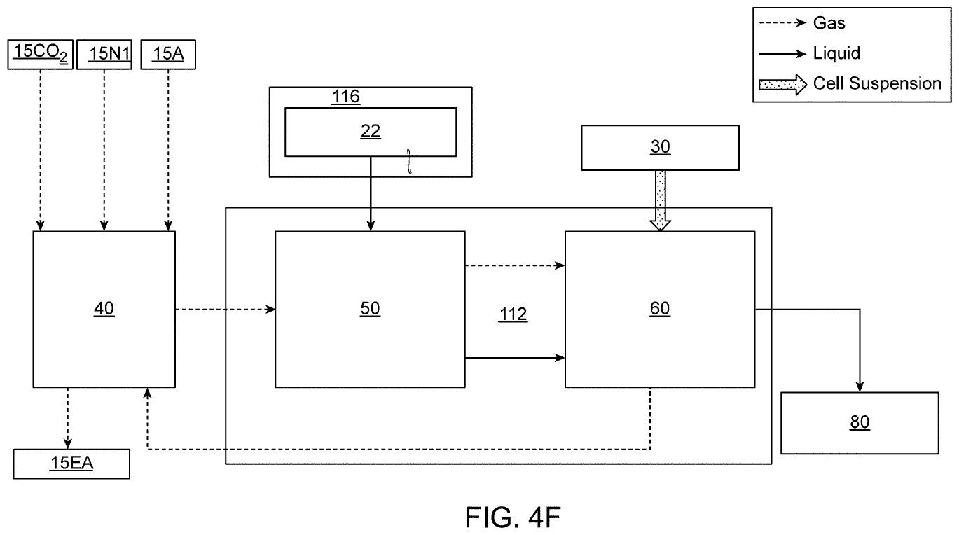

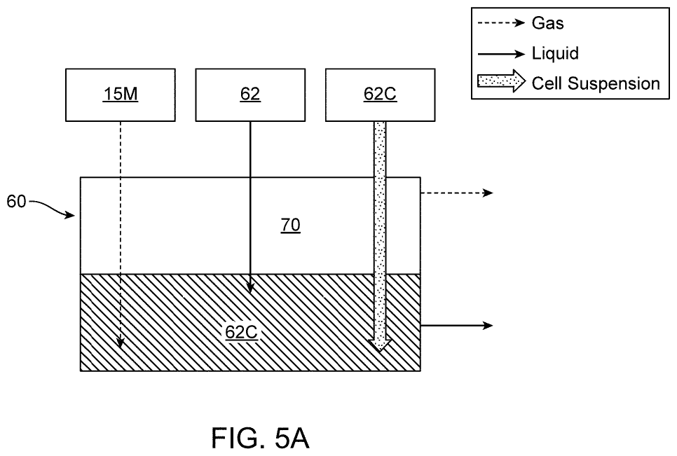

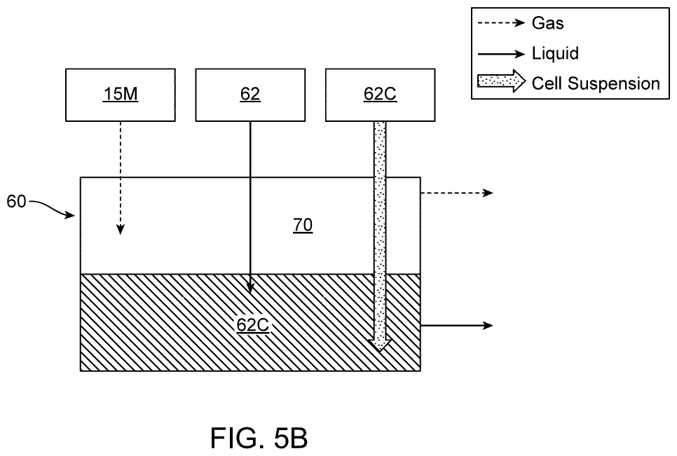

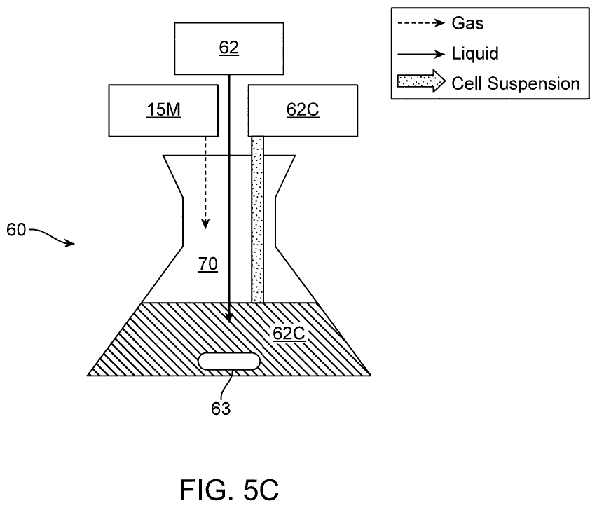

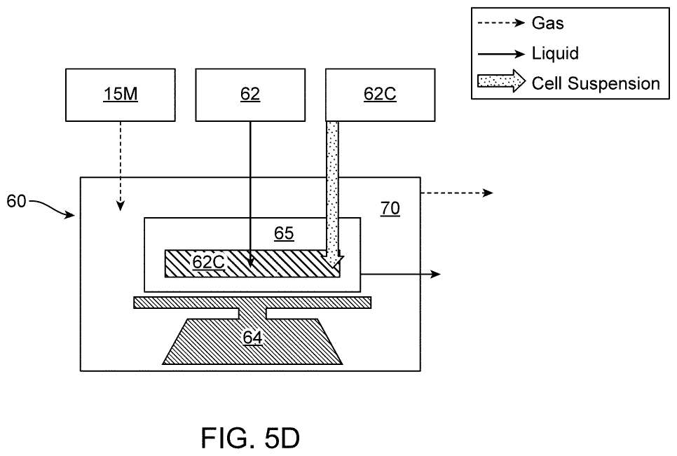

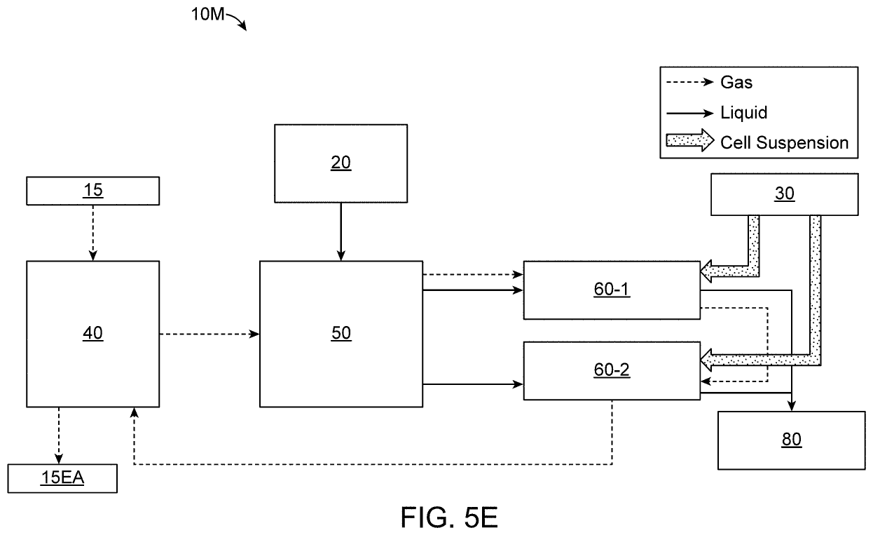

FIGS. 4A-5F show aspects of an embodiment of a gas and liquid flow regulation system 10 for cell culture, with separate but intertwined paths for gas flow and liquid flow. These figures represent an alternative embodiment to the embodiment described above and depicted in FIGS. 1A-3G. A difference between the embodiments shown in FIGS. 4A-5F and those shown in FIGS. 1A-3G is that these alternative embodiments include a medium gassing chamber 50, as described below.

FIG. 4A shows an embodiment of a gas and liquid flow regulation system for cell culture, with a focus on a gas flow aspect of the system; FIG. 4B shows an embodiment of a gas and liquid flow regulation system for cell culture, with a focus on a liquid flow aspect of the system. FIG. 4C shows an embodiment of a gas and liquid flow regulation system for cell culture, including the both the gas flow and liquid flow aspects of the system, effectively combining aspects of both FIGS. 4A and 4B.

FIGS. 4A-4C provide views of a gas and liquid flow regulation system 10 for cell culture at a basic level, with a focus on gas and liquid flow paths through component containers or chambers within the system. Gas containers or chambers include gas sources 15, a pressurizable gas mixing chamber 40, a medium gassing-chamber 50, and a cell culture compartment 60. These various components are connected by segments of a gas flow path, as defined by an originating source or container, and a receiving container or vent. As shown in FIG. 4C, culture medium gassing chamber 50 includes gas entry from one or more gas sources and a liquid entry from a medium source container 20; the culture medium gassing chamber is positioned between the pressurizable gas mixing chamber 40 and cell culture compartment 60. Both gas and liquid flow from medium gassing chamber 50 into the cell culture compartment 60.

Turning first to gas flow path 100 and its segments and container portions per FIG. 1A and FIG. 4C. Gas from one or more gas sources 15 is injected into pressurizable gas mixing chamber 40 by way of gas flow path segment 102. Mixed pressurized gas from pressurizable gas mixing chamber 40 flows into medium gassing chamber 50 by way of gas flow path segment 103. Pressurized gas from medium gassing chamber 50 flows into cell culture compartment 60 by way of flow path segment 104. Pressurized gas from cell culture compartment 60 flows back to pressurizable gas mixing chamber 40 by way of gas flow path segment 105. Pressurized gas within pressurizable gas mixing chamber 40 can be released into the ambient environment by way of vent 106.

Each gas flow path segment (102-105) includes an exit port from its respective originating container and an entry port into the receiving container. The flow rate of gases through each path segment can be regulated at the respective originating exit port and/or the respective entry port. Gas flow path segments 103-105, collectively and typically, form a closed loop (i.e., closed to gas entry or exit. The total gas pressure within this loop, including the various containers within the loop (pressurizable gas mixing chamber 40, medium gassing chamber 50, and cell culture compartment 60) is substantially the same. In an optional configuration, cell culture compartment 60 may have a gas vent to the atmosphere. Gas pressure within components of the closed loop is generated by pressurizable gas mixing chamber 40. Pressure within pressurizable gas mixing chamber 40 is generated upstream by incoming gas from gas sources, which is compressed within the source or pushed by a pump or fan. Gas flowing out of pressurizable gas mixing chamber 40 may be further pushed downstream by gas pumps, as shown in FIG. 1E, as described below.

Turning next to the liquid flow path 200 portion of gas and liquid flow regulation system 10 per FIGS. 4B-4C. Embodiments of liquid flow path 200 include containers or compartments linked by flow path segments. Liquid cell culture medium is included within cell culture medium source container 20, medium gassing chamber 50, cell inoculum source container 30, cell culture compartment 60, and downstream culture container 80. Cell-free culture medium from medium source container 20 flows into medium gassing chamber 50 by way of liquid flow path segment 201. At any point after liquid cell culture medium has been exposed to atmospheric gas, the liquid cell culture medium typically contains dissolved gas, such as dissolved oxygen, which then travels within the liquid cell culture flow path.

Liquid cell culture medium from medium gassing chamber 50 flows into cell culture compartment 60 by way of liquid flow path segment 202. Cell-containing culture medium from cell culture inoculum container 30 flows into cell culture compartment 60 by way of liquid flow path segment 203. Liquid ell culture medium (either cell-free, partially cell-free, or cell-containing) flows from cell culture compartment 60 into downstream culture container 80 by way of liquid flow path segment 204. Liquid cell culture medium flow within the various liquid flow path segments and containers is driven by hydraulic pressure, typically generated by peristaltic pumps, as described further below.

FIG. 4D shows an embodiment of a gas and liquid flow regulation system 10 for cell culture in accord with FIG. 4A-4C, with a particular focus on sensors disposed within the gas and liquid flow paths. Sensors that can be deployed within gas spaces of the system may include any one or more of an oxygen sensor S-O2, a carbon dioxide sensor S-CO2, a water or humidity sensor S-H2O, a pressure sensor S-P. Gas spaces within gas flow regulation system 100 include containers, such as pressurizable gas sources 15, gas mixing chamber 40, medium gassing chamber 50, and cell culture compartment 60.

Sensors that can be deployed within liquid spaces of the system may include any one or more of a dissolved oxygen sensor S-DO, a carbon dioxide sensor S-CO2, a glucose sensor S-glucose, a lactate sensor S-lactate, a pH sensor S-PH, an oxidation-reduction potential (ORP) sensor S-ORP, and a temperature sensor S-Temp. Liquid spaces within liquid flow regulation system 200 include liquid cell culture medium source container 20, cell inoculum source container 30, cell culture compartment 60, and downstream collection container 80. Flow path segments between containers include flow path 102 from gas sources 15 to pressurizable gas mixing chamber 40, flow path 103 from pressurizable gas mixing chamber 40 to medium gassing chamber 50, flow path 104 from medium gassing chamber 50 to cell culture compartment 60, and flow path from cell culture compartment 60 back to pressurizable gas mixing chamber 40.

FIG. 4E shows an embodiment of a gas and liquid flow regulation system 10 for cell culture in accord with FIG. 4C, with a particular focus on and liquid pumps 210 and gas pumps 230 disposed within the liquid and gas flow paths, respectively. Gas pumps move gas through the gas flow by atmospheric or pneumatic pressure. Liquid pumps move liquid cell culture medium through the gas flow path by hydraulic pressure. As seen in FIG. 3, gas from the pressurizable gas mixing chamber 40 may be transferred into the medium gassing chamber 50 by pump moving gas at a fast rate and a pump moving gas at a slow rate. Typically, the slow rate pump directs gas into direct contact with the liquid medium, and the fast rate pump bypasses liquid, staying the in the gas portion of the medium gassing chamber.

FIG. 4F shows an embodiment of a gas and liquid flow regulation system 10 for cell culture disposed within a temperature-controlled cell culture incubator 12. In this embodiment, medium gassing chamber 50 and cell culture compartment 60 are positioned inside cell culture incubator 12. There is an advantage to minimizing the internal volume of cell culture incubator 12 in terms of the total space it occupies in a laboratory or manufacturing facility, and separate external components can be arranged and configured with flexibility. Controlling the temperature of cells in cell culture compartment 60 is essential, so there is an advantage to it being positioned inside the incubator. There is also an advantage to positioning the medium gassing chamber 50 within the incubator because feeding gas and liquid medium at the appropriate temperature into the cell culture compartment assures stability of temperature in the cell culture compartment as feed gas and liquid are delivered.

System components that may be positioned external to the incubator 12 include pressurizable gas mixing chamber 40, culture medium source container 20 (which may be advantageously refrigerated, and cell inoculum source container 30. There may be an advantage to pressurizable gas mixing chamber 40 being external to incubator 12 in that it allows that chamber to serve more than one incubator. Cell culture liquid medium source container 20 need not be at the incubator temperature before being brought into the incubator because medium gassing chamber 50 brings liquid medium to temperature before transferring it into the cell culture compartment. Containers such as gas sources 15 and downstream culture container 80 are sensibly positioned outside incubator 12.

The foregoing description of cell culture incubator 12 and the rationale for its configuration notwithstanding, other arrangements of gas and liquid flow components may be advantageous for various circumstances. In one example, cell culture compartment(s) 60 and/or medium gassing chamber 50 could be external to an incubator, albeit requiring a heating and/or jacketing configuration. All of these configurations are within the scope of the disclosed technology.

Some components of gas and liquid flow regulation system 10 are optional, including, in particular, culture medium gassing chamber 50 (as described above and shown in FIGS. 4A-4F), a chamber in which both gas and liquid pass through on their respective ways to cell culture compartment 60. This particular component 50 or an analogous component is not depicted in the gas and liquid flow regulation system embodiment 110 as described above or as shown in FIGS. 1A-1G. FIG. 2E is a diagram of a liquid flow aspect of embodiment gas and liquid flow regulation system 10 that includes an internal liquid cell culture medium container 120I; the figure does not include gas flow in the liquid container 120I, but it is an option.

Gas and liquid flow regulation systems 110 and 10 are both fully functional, and with exception of the culture medium gassing chamber component operate in very similar ways. The presence of a medium gassing chamber in system 10 and its absence in system 110 demonstrates its optionality. An optional component, such as cell a culture medium gassing container, may be fixedly installed within a cell culture instrument, it may be by-passable within the instrument, or it may be easily removable and installable.

The factors as to whether have a medium-gassing chamber may include owner preference or specifics of clinical production protocols, but typically relate, at least in part, to the scale of gas and liquid flow through the gas and liquid flow system. For example, if a volume of liquid cell culture medium moving through a cell culture compartment, as in a perfusion arrangement, is large relative to the total volume within a cell culture vessel (such as a bag), pre-warming the cell culture medium can protect the liquid cell culture medium in the cell culture vessel from being dropped by an influx of still refrigerated cell culture medium. Similarly, a relatively large volume of culture medium with a dissolved gas composition that differs significantly from a preferred dissolved gas composition can protect the dissolved gas composition within a cell culture compartment against deviating from the preferred dissolved gas composition. On the other hand, if through volumes of gas and liquid are relatively small, the functionality provided by a medium gassing chamber may not be necessary.

FIGS. 5A-5E are schematic views various embodiments of a cell culture compartment 60 as disposed within an alternative embodiment, as depicted in FIGS. 4A-4F, and ways in which atmospheric gas (gas-phase gas) and gas dissolved in liquid medium can achieve or approach equilibrium, as determined by the solubility of each individual gas within the liquid. FIG. 5E, below, is a block diagram of an embodiment that includes multiple cell culture compartments within a cell culture instrument.

An efficient exchange of gases within a cell culture compartment is advantageous for a cell culture process. For example, oxygen from the gas-phase gas needs to transfer into the liquid medium in order for cells in culture to have access to the oxygen.

More particularly, these figures show various embodiments of a cell culture vessel 61 (within cell culture compartment 60) and ways in which a cell culture within the vessel can be stirred or agitated in order to maintain a substantially uniformly distributed cell presence, medium composition, and dissolved gas composition during a cell culturing period.

FIG. 5A shows an embodiment of a gas and liquid flow regulation system 10 for cell culture, with a focus on an embodiment of a cell culture compartment 60 that includes a gas sparging element that extends into the liquid cell culture medium. Sparging as directly into liquid medium is a highly efficient way to introduce gas-phase gas into liquid medium.

FIG. 5B shows an embodiment of a gas and liquid flow regulation system 10 for cell culture, with a focus on an embodiment of a cell culture compartment 60 that has incoming gas flowing into a gas headspace above the liquid medium. FIG. 5C shows a cell culture flask with gas being introduced into a gas headspace above the liquid medium, with the liquid medium (and cells contained therein) being stirred or mixed by a magnetic stir bar.

FIG. 5D shows an embodiment of a gas and liquid flow regulation system 10 for cell culture, with a focus on an embodiment of a cell culture compartment 60 that has a gas-permeable cell culture bag disposed within a gas space 70. Mixing of cell culture medium within the bag is created by the action of a platform rocker that supports the cell culture bag. In one example of an appropriate cell culture bag is provided by PermaLife Cell Culture bags by OriGen Biomedical (Austin Tex.).

It is advantageous for a cell culture that cells be substantially uniformly distributed as this gives individual cells within the population to have substantially equal access to nutrients in the liquid cell culture medium. It is advantageous for a cell culture that culture medium composition to be substantially uniform throughout the cell culture vessel as this gives individual cells within the population to have substantially equal access to the same level of nutrients in the liquid cell culture medium. It is advantageous for a cell culture's dissolved gases be uniformly distributed throughout the liquid cell culture medium volume, as this provides that individual cells within the cultured cell population to be exposed to the same local composition of dissolved gases.

FIG. 5E shows various aspects of an embodiment of a gas and liquid flow regulation system 10M for cell culture, with separate but intertwined paths for gas flow and liquid flow, that includes multiple cell culture compartments. The multiple cell culture compartments are served by a single medium gassing chamber 50, and by a single pressurized gas mixing chamber 40 provides an approach to scaling up cell culture volume within a single system.

Gas containers or chambers include gas sources 15, a pressurizable gas mixing chamber 40, a medium gassing-chamber 50, multiple cell culture compartments 60. These various components are connected by segments of a gas flow path, as defined by (b) an originating source or container, and (b) a receiving container or vent.

Gas from one or more gas sources 15 is injected into pressurizable gas mixing chamber 40 by way of gas flow path segment 102. Mixed pressurized gas from pressurizable gas mixing chamber 40 flows into medium gassing chamber 50 by way of gas flow path segment 103. Pressurized gas from medium gassing chamber 50 flows into a first of the multiple cell culture compartments 60 by way of flow path 104. Pressurized gas flows from the first of the multiple cell culture compartment 60 into a second cell culture compartment by way of a flow path 104-2 in a serial gas flow pattern. Pressurized gas from the second or final of the multiple cell culture compartments 60 returns back to pressurizable gas mixing chamber 40 by way of gas flow path segment 105. Pressurized gas within pressurizable gas mixing chamber 40 can be released into the ambient environment by way of vent 106.

Each gas flow path segment (102-105) includes an exit port from its respective originating container and an entry port into the receiving container. The flow rate of gases through each path segment can be regulated at the respective originating exit port and/or the respective entry port. Gas flow path segments 103-105, collectively, form a closed loop (i.e., closed to gas entry or exit. The total gas pressure within this loop, including the various containers within the loop (pressurizable gas mixing chamber 40, medium gassing chamber 50, and cell culture compartments 60) is substantially the same. Gas pressure within components of the closed loop is generated by pressurizable gas mixing chamber 40. Pressure within pressurizable gas mixing chamber 40 is generated upstream by incoming gas from gas sources, which is either compressed within the source or pushed by a pump or fan.

Turning next to the liquid flow path 200 portion of gas and liquid flow regulation system 10M. Embodiments of liquid flow path 200 include containers or compartments linked by flow path segments. Liquid cell culture medium is included within cell culture medium source container 20, medium gassing chamber 50, cell inoculum source container 30, cell culture compartments 60, and downstream culture container 80. Cell-free culture medium from medium source container 20 flows into medium gassing chamber 50 by way of liquid flow path segment 201. Liquid cell culture medium from medium gassing chamber 50 flows into cell culture compartment 60 by way of liquid flow path segment 202. Cell-containing culture medium from cell culture inoculum container 30 flows into cell culture compartments 60 by way of liquid flow path segments 203. Cell culture medium (either cell-free, partially cell-free, or cell-containing) flows from cell culture compartment 60s into downstream culture container 80 by way of liquid flow path segment 204. Liquid cell culture medium flow within the various liquid flow path segments and containers is driven by hydraulic pressure, typically generated by peristaltic pumps.

### Methods and Cell Types

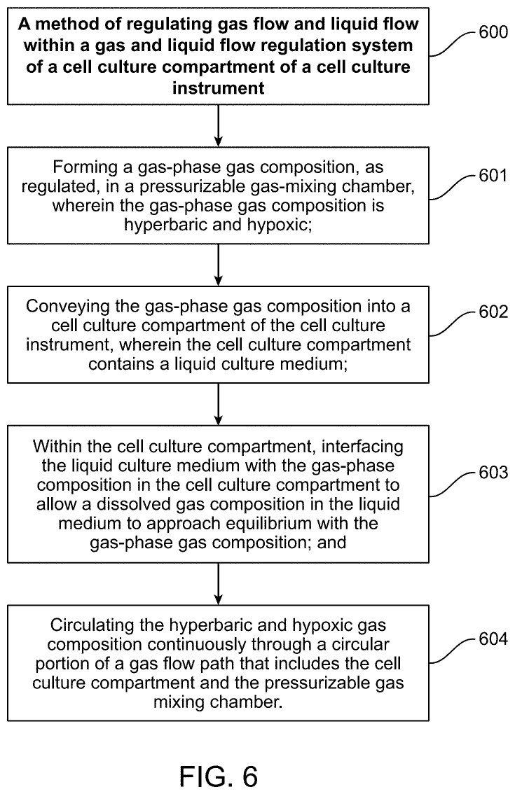

FIG. 6 is a flow diagram of a method 600 of regulating gas flow and liquid flow within a gas and liquid flow regulation system of a cell culture compartment of a cell culture instrument. Method steps recited 601-604, as recited below, are not intended to be indicative of the order in which steps may be performed.

Step 601 includes forming a gas-phase gas composition, as regulated, in a pressurizable gas-mixing chamber within the cell culture instrument, wherein the gas-phase gas composition is hyperbaric and hypoxic.

Step 602 includes conveying the gas-phase gas composition into a cell culture compartment, wherein the cell culture compartment contains a liquid culture medium.

Step 603 includes, within the cell culture compartment, interfacing the liquid culture medium with the gas-phase composition in the cell culture compartment to allow a dissolved gas composition in the liquid medium to approach equilibrium with the gas-phase gas composition.

Step 604 includes circulating the hyperbaric and hypoxic gas composition continuously through a circular portion of a gas flow path within the cell culture instrument that includes the cell culture compartment and the pressurizable gas mixing chamber.

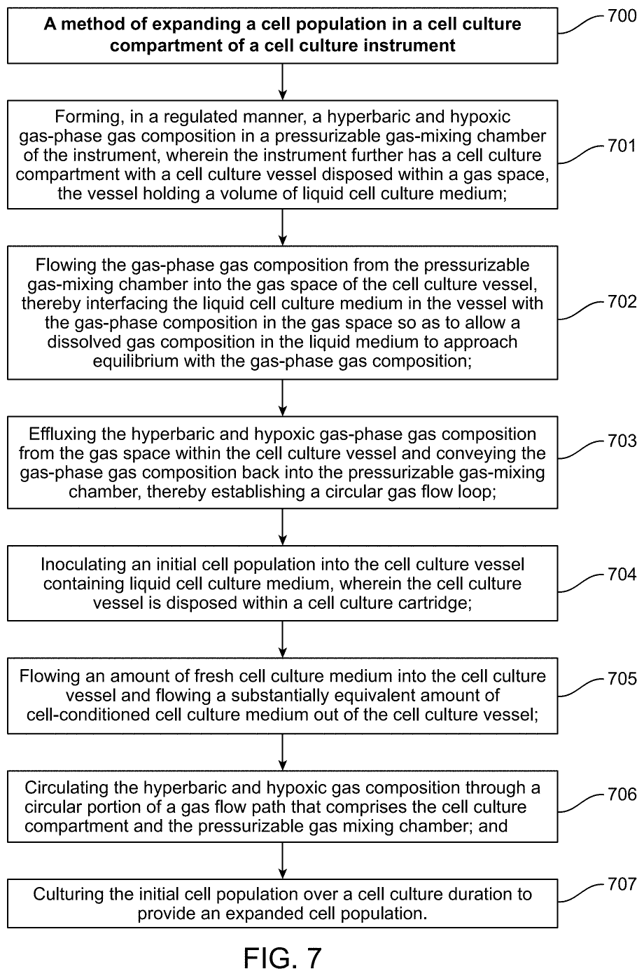

FIG. 7 is a flow diagram of a method 700 of expanding a cell population in a cell culture instrument of a cell culture instrument. Method steps 701-707, as recited below, are not intended to be indicative of the order in which steps may be performed. Expanding a cell population refers to starting a cell culture with an inoculating cell culture density and allowing it to grow to a higher cell density.

Step 701 includes forming, in a regulated manner, a hyperbaric and hypoxic gas-phase gas composition in a pressurizable gas-mixing chamber of the instrument, wherein the instrument further having a cell culture compartment with a cell culture vessel disposed within a gas space, and the cell culture vessel holding a volume of liquid cell culture medium. In particular embodiments, the vessel is a gas permeable cell culture bag.

Step 702 includes flowing the gas-phase gas composition from the pressurizable gas-mixing chamber into the gas space of the cell culture compartment, thereby interfacing the liquid culture medium in the cell culture vessel with the gas-phase composition in the gas space so as to allow a dissolved gas composition in the liquid medium to approach equilibrium with the gas-phase gas composition. In particular embodiments, gas-phase gas and gas dissolved in cell culture medium interface across a surface of a gas permeable cell culture bag.

Step 703 includes effluxing the hyperbaric and hypoxic gas-phase gas composition from the gas space within the cell culture vessel and conveying the gas-phase gas composition back into the pressurizable gas-mixing chamber, thereby establishing a circular gas flow loop.

Step 704 includes inoculating an initial cell population into the cell culture bag (such as a gas permeable cell culture bag) containing liquid cell culture medium, wherein the cell culture vessel is disposed within a cell culture cartridge.

Step 705 includes flowing an amount of fresh cell culture medium into the cell culture bag and flowing a substantially equivalent amount of cell-conditioned cell culture medium out of the cell culture bag. Cell-conditioned cell culture medium refers to medium in which cells have been grown, and thereby altered the composition of the medium.

Step 706 includes circulating the hyperbaric and hypoxic gas composition through a circular portion of a gas flow path that comprises the cell culture compartment and the pressurizable gas mixing chamber.

Step 707 includes culturing the initial cell population over a cell culture duration to provide an expanded cell population.

Aspects of a Cell Culture Process as Operated within a Cell Culture Instrument

### Tables 1A-1B: Gas Flow Regulation Operating Rules

Tables 1A-1B summarize gas flow regulation operating rules. The information provided in Table 1A and Table 1B are the very much the same, but organized differently. Table 1A relates to atmospheric of gas composition conditions within an incubator and system responses to conditions related to pressure level, oxygen level, or carbon dioxide level. Table 1B focuses on gas flow of nitrogen, air, carbon dioxide, and venting, what these gas flow operations are responsive to, and their affects. Set points refer to the sensed level of a gas that triggers a gas flow response.

TABLE 1A
Atmospheric Conditions within an
Incubator and Responses Thereto

| | |
|---|---|
| Atmospheric Condition | Gas Flow Response |

Pressure Level

| | |
|---|---|
| Pressure is below set point | Inject nitrogen |
| Pressure is above set point | Release atmosphere through vent |
| | (stop any ongoing injection of nitrogen) |

Oxygen Level

| | |
|---|---|
| Oxygen is below set point | Inject air |
| Oxygen is above set point | Inject nitrogen |

Carbon Dioxide Level

| | |
|---|---|
| CO2 is below set point | Inject CO2 |
| CO2 is above set point | stop injecting CO2, inject nitrogen |

TABLE 1B
Gas Flow Actions in Response to Atmospheric
Conditions within an Incubator

| | In response to a sensed | |
|---|---|---|
| Gas Flow | atmospheric condition | |
| Action | within an incubator | Effect |
| Nitrogen | Any of these three conditions . . . | Increase nitrogen |
| injection | pressure is below the pressure | flow |
| | set point | |
| | O2 is above O2 set point | |
| | CO2 is above CO2 set point | |
| | pressure is compliant with or above | Decrease or cease |
| | pressure set point | nitrogen flow |
| Air injection | Only when O2 is below the O2 | Increase air flow |
| | set point | |
| | When O2 is compliant with O2 | Decrease or cease |
| | set point | air flow |
| CO2 injection | Only when CO2 is below the CO2 | Increase CO2 flow |
| | set point | |
| | CO2 is compliant with CO2 set | Decrease or cease |
| | point | CO2 flow |
| Gas release | When pressure is above pressure | Controlled gas |
| through a vent | set point | release |
| | When pressure is compliant with | Decrease or cease |
| | pressure set point | gas release |

### Cells and Cell Culture Process Parameters

Embodiments of the invention include the tracking and application of various types of culture process data to methods of operating the gas and liquid flow systems provided herein, as hosted within a cell culture incubator. These data are used to monitor the progress of a cell culture process, and, in some instances, to provide feedback control of the process in real time. Some of the data are derived from real time monitoring; some of the data are derived from episodic sampling, and thus are not gathered in real process time, but rather intermittently. Episodic data, however, can be added into the totality of data that depict the cell culture process.

These process data include sensors for gases in gas phase and gases in liquid phase, within cell culture medium. These data further include particular nutritional components of cell culture medium (e.g., glucose, glutamine) as well as metabolic compounds produced by cells during a cell culture process.

These process data further include gas and liquid flow rates occurring within the cell culture incubator. Liquid flow rates are tracked in terms of absolute volume flow rate as well as flow rates relative to the working liquid volume of a cell culture container within the incubator.

These process data further include cell density values, in any of the various forms enumerated elsewhere herein. In some embodiments, cell density data can be captured in real time, but more typically, are gathered from intermittent sampling of cell culture medium from the cell culture container.

Cell culture data from sensors within the liquid of the liquid flow system, such as medium components, metabolites, and cell density may be combined with relative volumetric flow rates to derive cell-specific process parameters such cell-specific nutrient consumption rates, operating cell days/ml, or the reciprocal value of cell-specific medium volume consumption (e.g., nl/cell/day).

In some embodiments, culturing a cell population within the cell culture compartment may refer to expanding a population of hematopoietic cells within a workflow of preparing hematopoietic stem cells for transplantation into a patient. In some embodiments, culturing a cell population within the cell culture compartment may refer to expanding a population of autologous cells from a patient to prepare an enhanced cell population for an immunotherapy procedure. In particular embodiments, chimeric antigen receptor (CAR) T cells may be expanded in cell culture instrument 112. Merely by way of example, CAR-T cells may include CAR-NK or CAR-Treg cells. Further there may be a host of other modifications beyond CAR-based edits that would improve cell culture performance, improve performance of a cell-based product, or provide an alternative to targeting. In general, however, the provided technology may be applied to any type of cell, particularly any mammalian cell or human-derived cell.

### Cell Culture as a Clinical Manufacturing Process (Batch, Perfusion)

In some embodiments, the method of regulating gas flow and liquid flow within a cell compartment further includes culturing a cell population within the cell culture compartment. In some of these embodiments, culturing the cell population within the cell culture compartment includes expanding the cell population within a clinical manufacturing process workflow. A clinical manufacturing process may include any of a batch process, a fed-batch process, or a continuous culture process.

A batch process is one in which a cell culture run is terminated at a point of culture exhaustion or at a point determined by an operator or by a predetermined culture plan. A fed batch process is one in which one or more medium component solutions are added to a cell culture container in order to extend the life of the batch process. A continuous process, typically a perfusion process, is one in which cells in the cell culture container can reach a higher cell density than in a batch process. Perfusion refers to cell culture medium passing through (entering, exiting) a cell culture container (such as a cell culture bag) while retaining the cells. One example of a way to retain cells, while flowing medium out of a cell culture container in a perfusion manner, is to include a tangential flow filter in the medium exit flow path; this allows liquid medium to pass through the filter and move downstream, and for cells to be retained.

### Embodiment List

Sets of embodiments of the provided technology include the following: (1) a gas and liquid flow regulation system, (2) a method of regulating gas flow, (3) a method of regulating gas and liquid flow, (4) a method of expanding a cell population, and (5) a cell culture instrument.

### A First Set of Embodiments: A Gas and Liquid Flow Regulation System

1. A first embodiment of the technology is directed to a gas flow and liquid flow regulation system for a cell culture instrument that includes: a pressurizable gas-mixing chamber including multiple gas injection ports operably connected to multiple gas sources; a cell culture compartment including (a) a cell culture vessel that can hold a liquid medium for cell culture and (b) a gas space, wherein the liquid medium and the gas space meet at an interface; and a gas flow system including a circular flow path portion, the circular portion including (a) a first gas flow path segment from the pressurizable gas-mixing chamber to the cell culture compartment; (b) a second gas flow path segment from cell culture compartment back to the pressurizable gas-mixing chamber, wherein the gas flow system is adaptable to provide a hyperbaric and hypoxic atmospheric condition within the cell culture compartment.

2. The gas and liquid flow regulation system of embodiment 1 wherein gas and liquid flow regulation system is disposed within a temperature-controlled incubator portion of the cell culture instrument, and wherein the cell culture instrument is disposed within a housing.

3. The gas and liquid flow regulation system of embodiment 2 wherein the cell culture instrument includes a non-temperature controlled space within the housing.

4. The gas and liquid flow regulation system of embodiment 1 wherein the circular flow path portion of the gas flow system wherein the hyperbaric atmospheric condition and hypoxic atmospheric condition is substantially uniform throughout the circular flow path portion.

5. The gas and liquid flow regulation system of embodiment 4 wherein the hyperbaric atmospheric condition within the gas flow system is supported by an application of pneumatic pressure from the multiple gas sources.

6. The gas and liquid flow regulation system of embodiment 4 wherein the circular portion of the gas flow path portion of the gas flow system flows continuous when the gas flow system is in operation.

7. The gas and liquid flow regulation system of embodiment 1 wherein the pressurizable gas mixing chamber includes a vent to an atmospheric space that is external to the gas and liquid flow system.

8. The gas and liquid flow regulation system of embodiment 1 wherein the wherein the cell culture compartment includes a cartridge, the cartridge including: (a) a gas entry port and a liquid medium entry port, wherein the gas entry port is connected to the pressurizable gas-mixing chamber, and wherein the liquid medium entry port is connected to a liquid medium source container, (b) a gas exit port that directs gas flow to return to the pressurizable gas-mixing chamber, and (c) a liquid medium exit port, directing liquid flow to a downstream culture container.

9. The gas and liquid flow regulation system of embodiment 8 wherein the cartridge is cylindrical and configured to be insertable into and removable from the cell culture instrument that hosts the gas and liquid flow system.

10. The gas and liquid flow regulation system of embodiment 8 wherein the cylindrical cartridge hosts a gas-permeable cell culture bag that accommodates the liquid medium and a population of cultured cells within the medium.

11. The gas and liquid flow regulation system of embodiment 8 wherein the interface between the liquid medium and the gas space within the cylindrical cartridge includes a gas-permeable cell culture bag surface.

12. The gas and liquid flow regulation system of embodiment 8 includes (a) separate entry and exit ports for liquid medium, through which liquid medium can circulate, and (b) separate entry and exit ports for gas, through which gas may circulate.

13. The gas and liquid flow regulation system of embodiment 1 wherein a flow rate of gas through the circular portion of the gas flow path is regulatable and is responsive in a feedback manner to sensed gas-phase gas data originating from within the circular portion of the gas flow path.

14. The gas and liquid flow regulation system of embodiment 13 wherein the gas sensed data are delivered by one or more gas sensors disposed in the pressurizable gas mixing chamber or the cell culture compartment.

15. The gas and liquid flow regulation system of embodiment 13 wherein the sensed data originating from within the circular portion of the gas flow path relates to a composition of gas in atmospheric phase and/or gas dissolved in liquid culture medium.

16. The gas and liquid flow regulation system of embodiment 1 wherein a flow rate of gas through the circular portion of the gas flow path is regulatable and is responsive in a feedback manner to sensed dissolved gas data originating from within the circular portion of the gas flow path.

17. The gas and liquid flow regulation system of embodiment 16 wherein a flow rate of gas through the circular portion of the gas flow path is regulatable and is responsive in a feedback manner to sensed data originating from a liquid flow path within the gas flow and liquid flow regulation system.

18. The gas and liquid flow regulation system of embodiment 16 wherein a flow rate of gas through the circular portion of the gas flow path is controlled by one or more pneumatic flow rate mechanisms within the gas flow path.

19. The gas and liquid flow regulation system of embodiment 1 wherein the pressurizable gas-mixing chamber includes a gas entry port from the one or more gas sources and a regulatable vent to allow gas release from the pressurizable gas-mixing chamber.

20. The gas and liquid flow regulation system of embodiment 1 further including a cell culture inoculum source container configured to contain cells suspended in a liquid cell culture medium, wherein the inoculum source is operably connected to the cell culture vessel.

21. The gas and liquid flow regulation system of embodiment 1 further including a liquid flow regulation system including a liquid flow path having: (a) a liquid flow path segment from the liquid cell culture medium source container into the cell culture compartment; (b) a liquid flow path segment from the inoculum source container into the cell culture compartment; and (c) a liquid flow path segment from the cell culture compartment into a downstream cell culture collection container.

22. The gas and liquid flow regulation system of embodiment 21 wherein a flow rate of liquid through the liquid flow system is independent of the flow rate of gas through the gas flow system.

23. The gas and liquid flow regulation system of embodiment 21 further including by one or more hydraulic flow rate mechanisms.

24. The gas and liquid flow regulation system of embodiment 23 wherein the one or more hydraulic flow rate mechanisms includes one or more peristaltic pumps or hydraulic flow valves within the liquid flow path.

25. The gas and liquid flow regulation system of embodiment 1 wherein a flow rate of liquid medium through from the medium source container and through the cell culture compartment is regulatable and responsive in a feedback manner to sensed dissolved analyte data.

26. The gas and liquid flow regulation system of embodiment 25 wherein the dissolved analyte data include any one or more of dissolved gas levels, liquid medium components, cellular metabolites of liquid medium components, or a physicochemical property of the liquid.

27. The gas flow and liquid flow regulation system of embodiment 1 further including a culture medium gassing chamber comprising a gas entry and a liquid entry, wherein the culture medium gassing chamber is positioned between the pressurizable gas mixing chamber and the cell culture compartment, wherein both gas and liquid within the medium gassing chamber flow into the cell culture compartment.

### A Second Set of Embodiments: Method of Regulating Gas Flow and Liquid Flow

28. A second embodiment of the technology is directed to a method of regulating gas flow and liquid flow within a gas and liquid flow regulation system of a cell culture compartment of a cell culture instrument, the method including: (a) forming a gas-phase gas composition, as desired, in a pressurizable gas-mixing chamber, wherein the gas-phase gas composition includes a hyperbaric atmospheric pressure and a hypoxic oxygen partial pressure; (b) conveying the gas-phase gas composition into the cell culture compartment, wherein the cell culture compartment contains a liquid culture medium; (c) within the cell culture compartment, interfacing the liquid culture medium with the gas-phase composition in the cell culture compartment so as to allow a dissolved gas composition in the liquid medium to approach equilibrium with the gas-phase gas composition; and (d) circulating the hyperbaric and hypoxic gas composition through a circular portion of a gas flow path that includes the cell culture compartment and the pressurizable gas mixing chamber.

29. The method of embodiment 28 wherein circulating the hyperbaric and hypoxic gas-phase composition through the circular portion of gas flow path includes circulating the gas continuously.

30. The method of embodiment 28 wherein a gas flow rate at which the desired hyperbaric and hypoxic gas-phase gas composition circulates through the circular portion of gas flow path is regulated.

31. The method of embodiment 30 wherein a gas flow rate limiting at which the hyperbaric and hypoxic gas-phase gas composition circulates through the circular portion of gas flow path is regulated at during the conveyance of the gas composition from the pressurizable gas mixing chamber to the cell culture compartment.

32. The method of embodiment 28 wherein a composition of gas effluxed from the cell culture compartment differs from the desired hyperbaric and hypoxic gas composition because of an action of cell culture metabolism within the cell culture compartment.

33. The method of embodiment 28 wherein circulating the hyperbaric and hypoxic gas-phase composition through the circular portion of gas flow path includes circulating the gas continuously.

34. The method of embodiment 28 wherein regulating gas and liquid flow through the cell culture compartment includes: (a) flowing liquid medium from a liquid medium source container into the cell culture compartment; and (b) flowing liquid medium from the cell culture compartment into a downstream liquid culture medium container.

35. The method of embodiment 34 wherein the cell culture compartment includes a cell culture bag, and wherein a flow rate of liquid medium into the cell culture bag is measured with an absolute volumetric term (ml/min).

36. The method of embodiment 34 wherein the cell culture compartment includes a cell culture bag, and wherein a flow rate of liquid medium into the cell culture bag is measured with a term that is relative to a standing volume within the cell culture bag (entry volume re relative to the standing volume).

37. The method of embodiment 34 wherein flowing liquid medium into the cell culture compartment includes flowing liquid medium into the cell culture compartment at an entry flow rate, wherein flowing liquid medium from the cell culture compartment includes a cell culture compartment exit flow rate, and wherein the entry flow rate and the exit flow rate are independently controllable.

38. The method of embodiment 37 wherein when the entry flow rate is greater than the exit flow rate, the volume of medium within the cell culture compartment increases, and when the entry flow rate is less than the exit flow rate, the volume of medium within the cell culture compartment decreases.

39. The method of embodiment 34 wherein flowing liquid medium into the cell culture compartment includes flowing liquid at an entry flow rate, wherein flowing liquid medium from the cell culture compartment includes an exit flow rate, and wherein when the entry flow rate and the exit flow rate are substantially equivalent, the liquid medium is perfusing cell culture medium through the cell culture compartment while maintaining a constant cell culture medium volume within the cell culture compartment.

40. The method of embodiment 28 wherein the rate of gas flow and the rate of liquid flow are regulated separately and independently of each other.

41. The method of embodiment 28 wherein, regulating a gas flow rate within the cell culture compartment includes responding in a feedback manner to sensory input from within the cell culture compartment, and wherein regulating the liquid flow rate within the cell culture compartment includes responding in a feedback manner to sensory input from the within the cell culture compartment.

42. The method of embodiment 41 wherein, gas flow responding in a feedback manner to sensory input from within the cell culture compartment includes responding to sensory input from any of atmospheric-based sensory input or liquid-based sensory input.

43. The method of embodiment 41 wherein, liquid flow may be any of continuous, episodic, or intermittent, and wherein sensory feedback from the cell culture compartment may be any of continuous, episodic, or intermittent.

44. The method of embodiment 41 wherein, liquid flow responding in a feedback manner to sensory input from within the cell culture compartment includes responding to sensory input from any of atmospheric-based sensory input or liquid-based sensory input.

45. The method of embodiment 45 further including regulating a gas flow rate and/or liquid flow rate, in a feedback manner, to sensory input from any portion of a gas flow path or a liquid flow path within a system includes any one or more of the pressurizable gas mixing chamber or a downstream culture container.

46. The method of embodiment 28 further including conveying a cell culture inoculum within a volume of cell culture medium into a cell culture gas-permeable cell culture bag within the cell culture compartment, thereby forming a nascent cell culture or contributing additional cells to an ongoing cell culture.

47. The method of embodiment 46 further including, after forming the nascent culture, releasing a volume of the ongoing cell culture from the cell culture vessel into a downstream culture container.

48. The method of embodiment 47 wherein a volume of cell culture content collected in the downstream culture container is used (a) as a sample to gain culture process data, (b) for collection of cellular product or supernatant product, or (c) for releasing, in a perfusion process, a liquid volume that corresponds to a liquid volume being added to the cell culture compartment.

49. The method of embodiment 28 wherein the cell culture compartment includes a cell culture vessel and a gas space, wherein the cell culture vessel holds the liquid culture medium, the method including interfacing of gas-phase gas within the gas space and gas in a dissolved phase within the liquid medium across a gas-liquid interface.

50. The method of embodiment 28 wherein equilibrating or substantially equilibrating a dissolved gas composition within the liquid culture medium includes facilitating transfer of gas-phase gas into medium-dissolved gas by movement of gas across a permeable cell culture bag surface.

51. The method of embodiment 28 wherein forming a desired hyperbaric and hypoxic gas composition includes designating an individual gas set point for any of a desired atmospheric-based parameter or a liquid-based parameter, as sensed by a gas-based sensor or a liquid-based sensor, respectively.

52. The method of embodiment 51 wherein designating an individual gas set point includes an operator inputting an atmospheric-based parameter or a liquid-based parameter into a control system for regulating gas flow and liquid flow.

53. The method of embodiment 51 wherein designating an individual gas set point includes operating, at least in part, by way of a predetermined workflow to a control system.

53. The method of embodiment 51 wherein designating an individual gas set point includes operating, at least in part, according to a workflow that is responsive to atmospheric-based sensor data feedback and/or liquid-based sensor data feedback.

55. The method of embodiment 51 wherein designating an individual gas set point includes operating, at least in part, according to a workflow that is informed by machine-learned experience.

56. The method of embodiment 28 wherein forming a desired hyperbaric and hypoxic gas composition includes designating a system set point for a desired atmospheric pressure, and in response to a sensed pressure below the pressure set point, injecting nitrogen into the pressurizable gas-mixing chamber.

57. The method of embodiment 28 wherein forming a desired hyperbaric and hypoxic gas composition includes designating a system set point for a desired atmospheric pressure, and in response to a sensed pressure above the pressure set point, releasing gas from the pressurizable gas-mixing chamber.

58. The method of embodiment 28 wherein forming a desired hyperbaric and hypoxic gas composition includes designating a system set point for a desired oxygen level, and in response to a sensed oxygen below the pressure set point, injecting air into the pressurizable gas-mixing chamber.

59. The method of embodiment 28 wherein forming a desired hyperbaric and hypoxic gas composition includes designating a system set point for a desired oxygen level, and in response to a sensed oxygen above the pressure set point, injecting nitrogen into the pressurizable gas-mixing chamber.

60. The method of embodiment 28 wherein forming a desired hyperbaric and hypoxic gas composition includes designating a system set point for a desired carbon dioxide level, and in response to a sensed carbon dioxide below the pressure set point, injecting carbon dioxide into the pressurizable gas-mixing chamber.

61. The method of embodiment 28 wherein forming a desired hyperbaric and hypoxic gas composition includes designating a system set point for a desired carbon dioxide level and in response to a sensed carbon dioxide above the pressure set point, injecting nitrogen into the pressurizable gas-mixing chamber.

62. The method of embodiment 28 further including culturing a cell population within the cell culture compartment.

63. The method of embodiment 62 wherein culturing the cell population within the cell culture compartment includes expanding the cell population within a clinical manufacturing process workflow.

64. The method of embodiment 63 wherein the clinical manufacturing process includes any of a batch process, a fed-batch process, or a continuous culture process.

65. The method of embodiment 62 wherein culturing the cell population within the cell culture compartment includes expanding a population of hematopoietic cells within a workflow of preparing hematopoietic stem cells for transplantation into a patient.

### A Third Set of Embodiments: A Method of Expanding a Cell Population

66. A third embodiment of the technology is directed to a method of expanding a cell population in a cell culture compartment of a cell culture instrument the method including: (a) forming a hyperbaric and hypoxic gas-phase gas composition in a pressurizable gas-mixing chamber of the instrument, wherein the instrument further includes a cell culture compartment includes a cell culture vessel disposed within a gas space, and wherein the vessel is holding a volume of liquid cell culture medium; (b) flowing the gas-phase gas composition from the pressurizable gas-mixing chamber into the gas space of the cell culture compartment, thereby interfacing the liquid culture medium in the cell culture bag with the gas-phase composition in the gas space so as to allow a dissolved gas composition in the liquid medium to approach equilibrium with the gas-phase gas composition; (c) effluxing the hyperbaric and hypoxic gas-phase gas composition from the gas space and conveying the gas-phase gas composition back into the pressurizable gas-mixing chamber, thereby establishing a circular gas flow loop; (d) inoculating an initial cell population into a gas permeable cell culture bag containing liquid cell culture medium, wherein the cell culture bag is disposed within a cell culture cartridge; (e) flowing an amount of fresh cell culture medium into the cell culture bag and flowing a substantially equivalent amount of cell-conditioned cell culture medium out of the cell culture bag; (f) circulating the hyperbaric and hypoxic gas composition through a circular portion of a gas flow path that includes the cell culture compartment and the pressurizable gas mixing chamber; and (g) culturing the initial cell population over a cell culture duration to provide an expanded cell population.

67. The method of embodiment 60 wherein a cell population to be expanded includes chimeric antigen receptor (CAR) T cells.

### A Fourth Set of Embodiments: A Gas and Liquid Flow Regulation System (Gas AND Liquid Flow, Both)

68. A fourth embodiment of the technology is directed to gas flow and liquid flow regulation system for a cell culture instrument including: (a) a pressurizable gas-mixing chamber including multiple gas injection ports operably connected to multiple gas sources; (b) a cell culture compartment including (1) a cell culture vessel that can hold a liquid medium for cell culture and (1) a gas space, wherein the liquid medium and the gas space meet at an interface; and c) a gas flow system including a circular flow path portion, the circular portion including (1) a first gas flow path segment from the pressurizable gas-mixing chamber to the cell culture compartment; (2) a second gas flow path segment from cell culture compartment back to the pressurizable gas-mixing chamber, wherein the gas flow system is adaptable to provide a hyperbaric and hypoxic atmospheric condition within the cell culture compartment; and (d) a liquid flow regulation system including a liquid flow path including (1) a liquid flow path segment from the liquid cell culture medium source container into the cell culture compartment; (1) a liquid flow path segment from the inoculum source container into the cell culture compartment; and (c) a liquid flow path segment from the cell culture compartment into a downstream cell culture collection container, wherein the flow path includes a perfusion cell culture process.

### A Fifth Set of Embodiments: An Instrument, a Housing, an Incubator, and a Gas and a Liquid Flow Regulation System

69. A fifth embodiment of the technology is directed to an instrument for cell culture including: (a) a housing; (b) a temperature controlled incubator disposed within the housing; (c) a gas flow and liquid flow regulation system for a cell culture instrument disposed within the incubator, (d) a pressurizable gas-mixing chamber disposed within the incubator; (e) a cell culture compartment disposed within the incubator, the compartment including (a) a cell culture vessel that can hold a liquid medium for cell culture and (b) a gas space, wherein the liquid medium and the gas space meet at an interface; and (f) a gas flow system disposed within the incubator, the gas flow system including a circular flow path portion disposed within the cell culture compartment, the circular portion including (1) a first gas flow path segment from the pressurizable gas-mixing chamber to the cell culture compartment; (2) a second gas flow path segment from cell culture compartment back to the pressurizable gas-mixing chamber, wherein the gas flow system is adaptable to provide a hyperbaric and hypoxic atmospheric condition within the cell culture compartment; and (g) a liquid flow regulation system disposed within the incubator, the liquid flow system including a liquid flow path, the flow path including: (1) a liquid flow path segment from the liquid cell culture medium source container into the cell culture compartment; (1) a liquid flow path segment from the inoculum source container into the cell culture compartment; and (3) a liquid flow path segment from the cell culture compartment into a downstream cell culture collection container.

### A Sixth Set of Embodiments: An Instrument, a Housing, an Incubator, and a Gas and a Liquid Flow Regulation System

70. A sixth embodiment of the technology is directed to an instrument for cell culture including a gas flow and liquid flow regulation system for a cell culture instrument including: a pressurizable gas-mixing chamber comprising multiple gas injection ports operably connected to one or more gas sources; a cell culture compartment comprising (a) a cell culture vessel that can hold a liquid medium for cell culture and (b) a gas space, wherein the liquid medium and the gas space meet at an interface; and a gas flow system comprising a circular flow path portion, the circular portion comprising (a) a first gas flow path segment from the pressurizable gas-mixing chamber to the cell culture compartment; (b) a second gas flow path segment from cell culture compartment back to the pressurizable gas-mixing chamber, wherein the gas flow system is adaptable to provide a hyperbaric atmospheric condition and an oxygen level that can be regulated to a value in the range of 2%-36% oxygen within the cell culture compartment.

Any one or more features or steps of any device or method embodiment of the inventions disclosed herein can be combined with any one or more other features of any other described embodiment of the invention, without departing from the scope of the invention. It should also be understood that the inventions are not limited to the embodiments that are described or depicted herein for purposes of exemplification, but are to be defined only by a fair reading of claims appended to the patent application, including the full range of equivalency to which each element thereof is entitled. Some theoretical considerations of the inventors have been advanced in this application; these theoretical considerations are offered strictly for the purpose of conveying concepts underlying the inventions, not to support any of the claims, all of which stand wholly independent of any theoretical considerations.

1. A gas flow and liquid flow regulation system for a cell culture instrument comprising:
a pressurizable gas-mixing chamber comprising multiple gas injection ports operably connected to multiple gas sources;
a cell culture compartment comprising (a) a cell culture vessel, wherein the cell culture vessel holds a liquid medium for cell culture; and (b) a gas space, wherein the liquid medium and the gas space meet at an interface within the cell culture compartment; and
a gas flow system comprising a circular flow path portion, the circular flow path portion comprising (a) a first gas flow path segment from the pressurizable gas-mixing chamber to the cell culture compartment; and (b) a second gas flow path segment from the cell culture compartment back to the pressurizable gas-mixing chamber, wherein the gas flow system provides a hyperbaric atmospheric condition and hypoxic atmospheric condition within the cell culture compartment.

2. The gas flow and liquid flow regulation system of claim 1, wherein the gas flow and liquid flow regulation system is contained within a temperature-controlled incubator portion of the cell culture instrument, and wherein the cell culture instrument is disposed within a housing.

3. (canceled)

4. The gas flow and liquid flow regulation system of claim 1, wherein the hyperbaric atmospheric condition and hypoxic atmospheric condition is substantially uniform throughout the circular flow path portion.

5. The gas flow and liquid flow regulation system of claim 4, wherein the hyperbaric atmospheric condition within the gas flow system is supported by an application of pneumatic pressure from the multiple gas sources.

6. The gas flow and liquid flow regulation system of claim 4, wherein the gas flow within the circular flow path portion of the gas flow system flows continuously when the gas flow system is in operation.

7. The gas flow and liquid flow regulation system of claim 1, wherein the pressurizable gas-mixing chamber comprises a vent to an atmospheric space that is external to the gas flow and liquid flow regulation system.

8. The gas flow and liquid flow regulation system of claim 1, wherein the cell culture compartment comprises a cartridge, the cartridge comprising:
a. a gas entry port and a liquid medium entry port, wherein the gas entry port is connected to the pressurizable gas-mixing chamber, and wherein the liquid medium entry port is connected to a liquid medium source container;
b. a gas exit port that directs gas flow to return to the pressurizable gas-mixing chamber; and
c. a liquid medium exit port, wherein the liquid medium exit port directs liquid flow to a downstream culture container.

9. (canceled)

10. The gas flow and liquid flow regulation system of claim 8, wherein the cartridge comprises the cell culture vessel, the cell culture vessel comprising a gas-permeable cell culture bag that is configured to accommodate the liquid medium and a population of cultured cells within the liquid medium.

11. The gas flow and liquid flow regulation system of claim 8, wherein the interface between the liquid medium and a gas space within the cartridge comprises a gas-permeable cell culture bag surface.

12. (canceled)

13. The gas flow and liquid flow regulation system of claim 1, wherein a flow rate of gas through the circular flow path portion of the gas flow path is regulatable and is responsive in a feedback manner to sensed gas-phase gas data originating from within the circular flow path portion of the gas flow path.

14. (canceled)

15. The gas flow and liquid flow regulation system of claim 13, wherein the sensed gas-phase data originating from within the circular flow path portion of the gas flow path relates to a composition of gas in atmospheric phase and gas dissolved in the liquid culture medium.

16. The gas flow and liquid flow regulation system of claim 1, wherein a flow rate of gas through the circular flow path portion of the gas flow path is regulatable and is responsive in a feedback manner to sensed dissolved gas data originating from within the circular flow path portion of the gas flow path.

17. The gas flow and liquid flow regulation system of claim 16, wherein a flow rate of gas through the circular portion of the gas flow path is regulatable and is responsive in a feedback manner to sensed data originating from a liquid flow path within the gas flow and liquid flow regulation system.

18. (canceled)

19. The gas flow and liquid flow regulation system of claim 1, wherein the pressurizable gas-mixing chamber comprises a gas entry port from the one or more gas sources and a regulatable vent to allow gas release from the pressurizable gas-mixing chamber.

20. The gas flow and liquid flow regulation system of claim 1, further comprising a cell culture inoculum source container configured to contain cells suspended in a liquid cell culture medium, wherein the inoculum source is operably connected to the cell culture vessel.

21. The gas flow and liquid flow regulation system of claim 1, further comprising a liquid flow regulation system comprising a liquid flow path comprising:
a. a liquid flow path segment from the liquid cell culture medium source container into the cell culture compartment;
b. a liquid flow path segment from the inoculum source container into the cell culture compartment; and
c. a liquid flow path segment from the cell culture compartment into a downstream cell culture collection container.

22. The gas flow and liquid flow regulation system of claim 21, wherein a flow rate of liquid through the liquid flow system is independent of the flow rate of gas through the gas flow system.

23. The gas flow and liquid flow regulation system of claim 21, further comprising one or more hydraulic flow rate mechanisms, wherein the one or more hydraulic flow rate mechanisms comprise one or more peristaltic pumps or hydraulic flow valves within the liquid flow path.

24. (canceled)

25. The gas flow and liquid flow regulation system of claim 1, wherein a flow rate of liquid medium through from the medium source container and through the cell culture compartment is regulatable and responsive in a feedback manner to sensed dissolved analyte data, wherein the dissolved analyte data comprise any one or more of dissolved gas levels, liquid medium components, cellular metabolites of liquid medium components, or a physicochemical property of the liquid.

26. (canceled)

27. The gas flow and liquid flow regulation system of claim 1, further comprising a culture medium gassing chamber comprising a gas entry and a liquid entry, wherein the culture medium gassing chamber is positioned between the pressurizable gas mixing chamber and the cell culture compartment, wherein both gas and liquid within the medium gassing chamber flow into the cell culture compartment.

28-70. (canceled)
